(19) United States
(12) Patent Application Publication
(10) Pub. No.: US 2026/0002124 A1
(43) Pub. Date: Jan. 1, 2026

(54) ENHANCING NEURONAL DIFFERENTIATION OF VENTRAL MIDBRAIN NEURAL PROGENITOR CELLS

(71) Applicant: Novo Nordisk A/S, Bagsvaerd (DK)

(72) Inventors: Djordje Djordjevic, London (GB); Jonathan Niclis, Koebenhavn (DK); Josefine Raagaard Christiansen, Copenhagen N (DK)

(21) Appl. No.: 18/878,471

(22) PCT Filed: Jun. 30, 2023
(86) PCT No.: PCT/EP2023/067996

§ 371 Date: Dec. 23, 2024

(30) Foreign Application Priority Data
Jul. 1, 2022 (EP) 22182681.1

(51) Int. Cl. C12N 5/0793

(52) U.S. Cl. CPC C12N 5/0619; C12N 2500/38; C12N 2501/119; C12N 2501/13; C12N 2501/41; C12N 2501/42; C12N 2501/727; C12N 2506/02; C12N 2533/52

(57) ABSTRACT

The present invention relates to a method for directing differentiation of ventral midbrain NSCs into neurons comprising contacting a cell population comprising ventral midbrain NSCs with an inhibitor of MEK signaling and an inhibitor of NOTCH signaling, wherein the ventral midbrain NSCs co-express the markers FOXA2, LMX1A, EN1, OTX2, 5 and SOX2.

### TECHNICAL FIELD

The present invention relates generally to the field of stem cells, such as human embryonic stem cells. Methods are provided for obtaining stem cell-derived neural cells. Specifically, methods are provided for obtaining stem cell-derived ventral midbrain neural cells for the treatment of Parkinson's disease.

### BACKGROUND

The prospect of using human pluripotent stem cells (hPSCs) in the treatment of various conditions seems very promising. Treatments include cell-replacement therapy of neurological conditions such as Parkinson's disease and stroke. For such treatments to become viable, however, it requires the development of in vitro methods to artificially produce the stem cell-derived products for their delivery to the central nervous system (CNS). The differentiation of hPSCs into defined cell types is a difficult process to control; in many cases the progeny generated from in vitro protocols are heterogenous. Typically, when differentiating into neural cells, the mixture of cell types produced includes neurons (and within this a variety of neuronal subtypes such as glutamatergic and dopaminergic neurons), glia, neural stem cells (NSCs) as well as other non-neuronal cells (e.g., meningeal stromal cells). Such heterogenous cultures are suboptimal for disease modelling studies or many cell replacement therapies.

Cellular replacement therapy for Parkinson's disease is a prime example. In Parkinson's disease, the A9 ventral midbrain dopaminergic neurons (vmDAs) are lost and arguably the sole cell type that must be transplanted to restore lost function. Presently, however, all academic scientific publications and all human clinical trials underway are transplanting a multipotent progenitor population that results in mixed populations of cells in vivo, in addition to vmDAs. This mixed population includes cell types such as proliferative NSCs, vascular leptomeningeal cells (VLMCs), non-vmDA neurons, and astrocytes. Non-dopaminergic neurons do not restore function in Parkinson's disease and some (i.e., serotonergic neurons) produce negative gain of function behaviors in patients as seen in clinical trials transplanting human fetal cells, and more generally these non-vmDAs carry unknown safety and efficacy risks. Consequently, there is a need to provide methods which ensure that patients are treated with ventral midbrain neurons or progenitors thereof.

It is therefore an object of the present invention to overcome the aforementioned challenges, in particular to provide methods which can direct the differentiation of cells into ventral midbrain neurons.

### SUMMARY

The object as outlined above is achieved by the aspects of the present invention. In addition, the present invention may also solve further problems, which will be apparent from the disclosure of the exemplary embodiments.

In a first aspect the present invention is provided a method comprising contacting a cell population comprising ventral midbrain NSCs with an inhibitor of MEK signaling and an inhibitor of NOTCH signaling. Specifically, the method is for directing differentiation of ventral midbrain NSCs into neurons, meaning that the developmental fate of the ventral midbrain neural cells is affected towards a certain outcome, i.e., neurons. However, the differentiation into neurons is not necessarily completed and the cells may not necessarily develop into the final fate during the method as disclosed herein. Preferably, the cells at the progenitor stage are directed towards differentiation into neurons in vitro, and suitable for administration to a patient wherein the further development from the progenitor stage in vivo results in a neuronal fate. The present inventors have found that the differentiation and manufacture of ventral midbrain neurons from a cell population, such as human PSCs, can be improved with the combined inhibition of the MEK signal pathway and the NOTCH signal pathway. In particular, the present inventors surprisingly found that the effects of MEK pathway inhibition can be enhanced by the combined addition of NOTCH pathway antagonists/inhibitors. Specifically, this combined inhibition reduces the proportion of late-stage proliferative cells (which represents off-target lineages), reduces the proportion of non-neural cells such as stromal cells, e.g., VLMCs, and increases the proportion of neurons. Importantly, the present inventors have found that the timing of inhibiting the MEK and NOTCH signaling relative to the developmental stage of the cells is influential on the outcome. In a preferred embodiment, the neural cells at time of inhibition of MEK and NOTCH signaling are at a stage wherein the neural cells comprise a mixture of neural stem cells, neuroblast intermediate precursor cells, and few neurons. In a preferred embodiment, the ventral midbrain NSCs prior to inhibition of MEK signaling are neurally induced, ventralized, and caudalized. In a preferred embodiment, at least 5% of the cell population comprising ventral midbrain NSCs co-express the markers FOXA2, LMX1A, EN1, OTX2, and SOX2. The cell population comprising such mixture of ventral midbrain neural cells may originate from any method. However, in an embodiment, the cell population is neurally induced, caudalized and ventralized prior to the inhibition of the MEK and NOTCH signaling. The cell population may be derived from pluripotent cells, such as PSCs. Accordingly, in a preferred embodiment a cell population of PSCs is neurally induced, ventralized, and caudalized according to well-known methods, such as exposing the PSCs to an inhibitor of SMAD protein signaling, an activator of SHH signaling, and an inhibitor of Wnt signaling, and further contacting the cell population with an activator of FGF signaling. A cell population of neurally induced cells, such as ventral midbrain NSCs, may be obtained according to a method of differentiation lasting e.g., 16 days from initiating neural induction, wherein the resultant cell population mainly comprises neural stem cells. Further culturing the cell population, such as for up to 8 days, allows more neural stem cells to further develop into neuroblast intermediate precursor cells yet still maintains a window of opportunity in respect to directing further differentiation of the ventral midbrain neural cells into neurons by contacting the cells with the inhibitor of MEK signaling and the inhibitor of NOTCH signaling. In the aforementioned method of neurally inducing PSCs, it has been found that cells cultured for too long, such as more than 28 days, may be difficult to harvest, i.a., due formation of an inseparable mesh of neurites.

A person skilled in the art will recognize that the best timing of contacting the cell population with the inhibitors may vary according to the specific protocol used to differentiate the PSCs into ventral midbrain NSCs. However, the present inventors have identified an expression profile of the cell population comprising ventral midbrain NSCs that provides an improved outcome of neurons. Accordingly, in a preferred embodiment, 40-60% of the cell population comprising ventral midbrain NSCs express ASCL1, 45-65% of the cell population comprising ventral midbrain NSCs express KI67, less than 10-15% of the cell population comprising ventral midbrain NSCs express INA, 2-5% of the cell population comprising ventral midbrain NSCs are INA+/SOX2−, and 80-95% of the cell population comprising ventral midbrain NSCs express SOX2 at the time of contacting the cell population with the inhibitor of MEK signaling and the inhibitor of NOTCH signaling. It follows that in an embodiment wherein a cell population is differentiated into a cell population comprising ventral midbrain NSCs, the cell population is allowed to differentiate until the cell population obtains the aforementioned expression profile at which point the cell population is contacted with the inhibitor of MEK signaling and the inhibitor of NOTCH signaling.

An in vitro cell population obtained according to the method may be used for the treatment of Parkinson's disease and may provide reduced surgical procedure time and cranial injections due to a higher purity cell product. In addition, improved product purity and reduced impurities are anticipated to provide an enhanced safety and potentially efficacy profile.

Another aspect of the present invention relates to an inhibitor of MEK signaling for use in the treatment of Parkinson's disease in a subject having been administered with a therapeutically effective amount of ventral midbrain NSCs. Specifically, the ventral midbrain NSCs co-express the markers FOXA2, LMX1A, EN1, OTX2, and SOX2. The present inventors contemplate that the effect of MEK and NOTCH inhibition demonstrated in vitro may be directly translated into an in vivo effect directing differentiation of administered ventral midbrain NSCs into neurons. Accordingly, following the administration of a cell population comprising ventral midbrain NSCs into a subject, such as by surgery, the present inventors believe that the administered cells may be further directed in differentiation towards neurons, thereby reducing the proportion of late-stage proliferative cells and non-neural cells such as VLMCs as well as increasing the proportion of neurons. In an embodiment, the inhibitor of MEK signaling is Mirdametinib, a clinically tested small molecule which acts as a MEK inhibitor and crosses the blood brain barrier. In an embodiment, the ventral midbrain NSCs are co-administered with the inhibitor of MEK signaling or treated prior to transplantation with inhibitors.

Similar aspects of the present invention relate to an inhibitor of MEK signaling, an inhibitor of NOTCH signaling and a cell population comprising ventral midbrain NSCs for use in combination in the treatment of Parkinson's disease, and a composition comprising a cell population comprising ventral midbrain NSCs, an inhibitor of MEK signaling, and an inhibitor of NOTCH signaling for the treatment of Parkinson's disease. Here, the present inventors contemplate that the ventral midbrain NSCs are co-administered with the inhibitor of MEK signaling and an inhibitor of NOTCH signaling. The ventral midbrain NSCs may or may not have been treated with an inhibitor of MEK signaling and an inhibitor of NOTCH signaling in vitro as according to the method disclosed herein prior to co-administration. It has been demonstrated that even short exposure of inhibitors of the MEK and NOTCH signaling directs a cell population comprising ventral midbrain NSCs towards neuronal fate, and a one-time co-administration of cells and inhibitors is likely sufficient to direct the further cell maturation in vivo, thus reducing the required dosage administration to the patient.

### BRIEF DESCRIPTION OF DRAWINGS

FIG. 1 shows a simplified schematic illustrating the stages of VM differentiation. The procedure begins with hPSCs (represented by a white object) that are differentiated to VM NSCs cells (represented by a vertically striped object) following neural induction, ventralization and caudalization. Compounds of the invention, MEK and/or NOTCH inhibitors, are administered after these steps and when VM IPCs (represented by a horizontal striped object) and VM Neurons (represented by a black object) have low expression. Inhibitor addition immediately following neural induction, ventralization and caudalization is termed “early administration”.

FIG. 2 shows a bar graph of the results of intracellular flow cytometry protein analysis of cell cultures at the time of beginning the early compound administration. Expression is shown for the NSC marker SOX2 and proliferative marker KI67. Expression is also shown for VM and floor plate lineage markers FOXA2, LMX1A and OTX2, as well as the VM floor plate IPC marker ASCL1 and neuronal marker INA. The graph displays the percentage of cells of the total viable cells.

FIG. 3 shows a bar graph of the results of intracellular flow cytometry protein analysis of cell cultures at day in vitro 22 after early administration. Results are shown of cultures with no intervention (Control, black bars), the administration of a MEK inhibitor (+MEKi, white bars), administration of a NOTCH inhibitor (+NOTCHi, narrow striped bars) and administration of a MEK and NOTCH inhibitor (+MEKi+NOTCHi, bold striped bars). Protein expression is shown for the NSC marker SOX2 and proliferative marker KI67, Neuronal marker INA and VM floor plate lineage markers FOXA2 and LMX1A. The graph displays the percentage of cells of the total viable cells.

FIG. 4 shows representative immunofluorescence images of cultures subject to early administration differentiated for an extended period of time to day in vitro 35. Results are shown from control conditions (A-B), following administration of a NOTCH inhibitor (C-D), following administration of a MEK inhibitor (E-F) and following administration of both a MEK and NOTCH inhibitor (G-H). Staining of all cell nuclei with DAPI (A, C, E, G) and SOX2 cells (B, D, F, H).

FIG. 5 shows representative immunofluorescence images of cultures subject to early administration differentiated for an extended period of time to day in vitro 35. Results are shown from control conditions (A-B), following administration of a NOTCH inhibitor (C-D), following administration of a MEK inhibitor (E-F) and following administration of both a MEK and NOTCH inhibitor (G-H). Staining of all neuronal fibers with the marker INA (A, C, E, G) and all proliferative cells with KI67 (B, D, F, H).

FIG. 6 shows representative immunofluorescence images of cultures subject to early administration differentiated for an extended period of time to day in vitro 35. Results are shown from control conditions (A-B), following administration of a NOTCH inhibitor (C-D), following administration of a MEK inhibitor (E-F) and following administration of both a MEK and NOTCH inhibitor (G-H). Staining of all cell nuclei with DAPI (A, C, E, G) and all dopamine neurons with tyrosine hydroxylase (B, D, F, H).

FIG. 7 shows representative immunofluorescence images of cultures subject to early administration differentiated for an extended period of time to day in vitro 35. Results are shown from control conditions (A-B), following administration of a NOTCH inhibitor (C-D), following administration of a MEK inhibitor (E-F) and following administration of both a MEK and NOTCH inhibitor (G-H). Staining of all ventral midbrain floor plate lineage cells with the marker LMX1A (A, C, E, G) and non-neuronal stromal cells stained with the marker COL1A1 (B, D, F, H).

FIG. 8 shows a simplified schematic illustrating the stages of VM differentiation. The procedure begins with hPSCs (represented by a white object) that are differentiated to VM NSCs cells (represented by a vertically striped object) following neural induction, ventralization and caudalization. Compounds of the invention, MEK and/or NOTCH inhibitors, are administered after these steps and after a period of time with no patterning factors which is when VM IPCs (represented by a horizontal striped object) and VM Neurons (represented by a black object) have increased in proportion and VM NSCs have decreased. Inhibitor addition after patterning factors and after time without patterning factors for neural induction, ventralization and caudalization is termed “late administration”.

FIG. 9 shows a bar graph of intracellular flow cytometry protein analysis of cell cultures at the time when late compound administration (MEKi, NOTCHi or MEKi+NOTCHi) was to be given to the cells; note these cells had not been exposed previously to any NOTCH or MEK inhibitors). Expression is shown for the NSC marker SOX2 and proliferative marker KI67. Expression is also shown for VM and floor plate lineage markers FOXA2, LMX1A and OTX2, as well as the VM floor plate IPC marker ASCL1 and neuronal marker INA. The graph displays the percentage of cells of the total viable cells.

FIG. 10 shows a bar graph of the results of intracellular flow cytometry protein analysis of cell cultures at the time of beginning the late compound administration. Expression is shown for the NSC marker SOX2 and proliferative marker KI67. Expression is also shown for VM and floor plate lineage markers FOXA2, LMX1A and OTX2, as well as the VM floor plate IPC marker ASCL1 and neuronal marker INA. The graph displays the percentage of cells of the total viable cells.

FIG. 11 shows a bar graph of intracellular flow cytometry protein analysis of cell cultures at the same time when late compound administration (MEKi, NOTCHi or MEKi+NOTCHi) is to be given, however, these cells had been exposed previously to NOTCH inhibitors as is traditionally done in the field and this is an undesirable profile and not preferable for the administration of MEKi and/or NOTCHi treatment. Expression is shown for the NSC marker SOX2 and proliferative marker KI67. Expression is also shown for VM and floor plate lineage markers FOXA2, LMX1A and OTX2, as well as the VM floor plate IPC marker ASCL1 and neuronal marker INA. The graph displays the percentage of cells of the total viable cells.

FIG. 12 shows representative immunofluorescence images of cultures subject to late administration differentiated for an extended period of time to day in vitro 40. Results are shown from control conditions (A-B), following administration of a NOTCH inhibitor (C-D), following administration of a MEK inhibitor (E-F) and following administration of both a MEK and NOTCH inhibitor (G-H). Staining of all cell nuclei with DAPI (A, C, E, G) and SOX2 cells (B, D, F, H).

FIG. 13 shows representative immunofluorescence images of cultures subject to late administration differentiated for an extended period of time to day in vitro 40. Results are shown from control conditions (A-B), following administration of a NOTCH inhibitor (C-D), following administration of a MEK inhibitor (E-F) and following administration of both a MEK and NOTCH inhibitor (G-H). Staining of all neuronal fibers with the marker INA (A, C, E, G) and all proliferative cells with KI67 (B, D, F, H).

FIG. 14 shows representative immunofluorescence images of cultures subject to late administration differentiated for an extended period of time to day in vitro 40. Results are shown from control conditions (A-B), following administration of a NOTCH inhibitor (C-D), following administration of a MEK inhibitor (E-F) and following administration of both a MEK and NOTCH inhibitor (G-H). Staining of all cell nuclei with DAPI (A, C, E, G) and all dopamine neurons with tyrosine hydroxylase (B, D, F, H).

FIG. 15 shows representative immunofluorescence images of cultures subject to late administration differentiated for an extended period of time to day in vitro 40. Results are shown from control conditions (A-B), following administration of a NOTCH inhibitor (C-D), following administration of a MEK inhibitor (E-F) and following administration of both a MEK and NOTCH inhibitor (G-H). Staining of all ventral midbrain floor plate lineage cells with the marker LMX1A (A, C, E, G) and non-neuronal stromal cells stained with the marker COL1A1 (B, D, F, H).

FIG. 16 shows protocols for neurally inducing, ventralizing and caudalizing a cell population of PSCs into ventral midbrain NSCs as well further directing differentiation of the cells towards neurons. In (A) the cell population is contacted with an inhibitor of MEK signaling and an inhibitor of NOTCH signaling at day 16. In (B) the cell population is allowed to further differentiate for a period of time prior to being contacted with an inhibitor of MEK signaling and an inhibitor of NOTCH signaling at day 22 with a deliberate absence of NOTCHi after patterning factors (i.e. SMAD inhibitors, SHH agonists, WNT agonists and FGF8s) are removed.

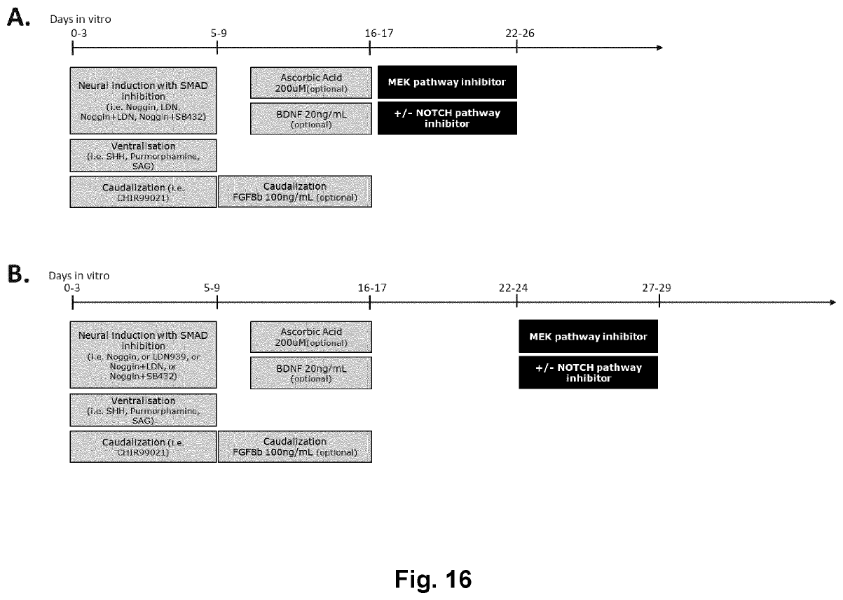

FIG. 17 shows a simplified schematic illustrating the stages of VM differentiation. The procedure begins with hPSCs (represented by a white object) that are differentiated to VM NSCs cells (represented by a vertically striped object) following neural induction, ventralization and caudalization. Compounds of the invention, NOTCH inhibitors, are administered after these steps and after a period of time with no patterning factors which is when VM IPCs (represented by a horizontal striped object) and VM Neurons (represented by a black object) have increased in proportion and VM NSCs have decreased. NOTCH Inhibitor addition after patterning factors and after time without patterning factors for neural induction, ventralization and caudalization is termed “late administration NOTCHi only”.

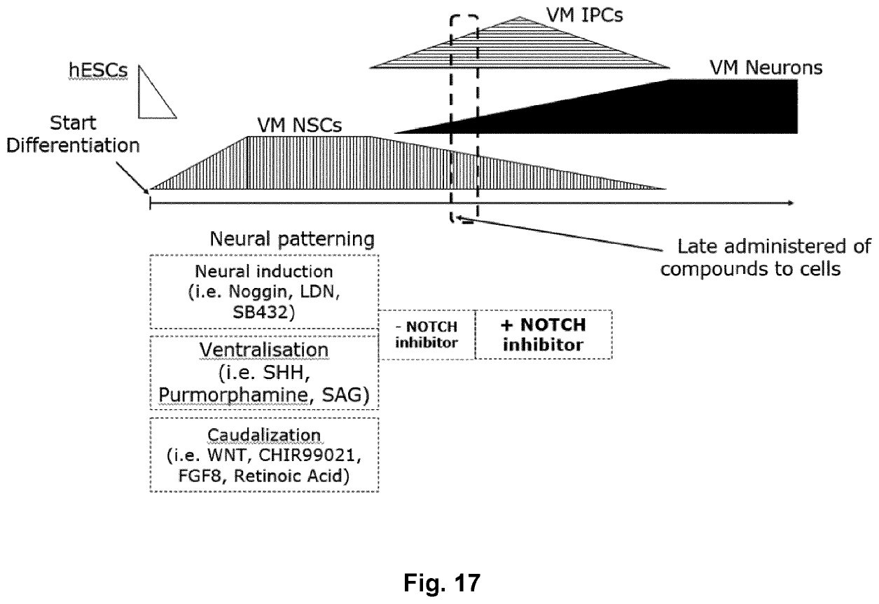

### DESCRIPTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed.

### Definitions

### General Definitions

As used herein, “a” or “an” or “the” can mean one or more than one. Unless otherwise indicated in the specification, terms presented in singular form also include the plural situation.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

### Stem Cells

By “stem cell” is to be understood an undifferentiated cell having differentiation potency and proliferative capacity (particularly self-renewal competence) but maintaining differentiation potency. The stem cell includes categories such as pluripotent stem cell, multipotent stem cell, unipotent stem cell and the like according to their differentiation potentiality.

As used herein, the term “pluripotent stem cell” (PSC) refers to a stem cell capable of being cultured in vitro and having a potency to differentiate into any cell lineage belonging to the three germ layers (ectoderm, mesoderm, endoderm), as well as lineage restricted undifferentiated stem cells that have lost the capacity to form some cell type(s), lineage(s) or developmental region(s) typically through genetic editing.

A pluripotent stem cell can be induced or isolated from a fertilized egg, somatic nuclear transfer embryo, germ stem cell, stem cell in a tissue, somatic cell and the like. Examples of the pluripotent stem cell (PSC) include embryonic stem cell (ESC), embryonic germ cell (EG cell), induced pluripotent stem cell (iPSC) and the like.

As used herein, the term “induced pluripotent stem cell” (also known as iPS cells or iPSCs) means a type of pluripotent stem cell that can be generated directly from cells that are not PSCs and have a nucleus. By the introduction of products of specific sets of pluripotency-associated genes non-pluripotent cells can be converted into pluripotent stem cells.

As used herein, the term “embryonic stem cell” means a pluripotent stem cell derived from the inner cell mass of a blastocyst. Pluripotent embryonic stem cells may also be derived from parthenotes as described in e.g., WO 2003/046141. Additionally, embryonic stem cells can be produced from a single blastomere or by culturing an inner cell mass obtained without the destruction of the embryo. Embryonic stem cells are available from given organizations and are also commercially available. Preferably, the methods and products of the present invention are based on human PSCs, i.e., stem cells derived from either human induced pluripotent stem cells or human embryonic stem cells, including parthenotes.

As used herein, the term “multipotent stem cell” means a stem cell having a potency to differentiate into plural types of tissues or cells, though not all kinds and is typically restricted to one germ layer. A neural stem cell is an example of a multipotent stem cell restricted to the neural lineage.

As used herein, the term “unipotent stem cell” means a stem cell having a potency to differentiate into only one particular cell type.

As used herein, the term “in vitro” means that the cells are provided and maintained outside of the human or animal body, such as in a vessel like a flask, multiwell or petri dish. It follows that the cells are cultured in a cell culturing medium.

As used herein, the term “non-native” means that the cells although derived from pluripotent stem cells, which may have human origin, is an artificial construct, that does not exist in nature. In general, it is an object within the field of stem cell therapy to provide cells, which resemble the cells of the human body as much as possible. However, it may never become possible to mimic the development which the pluripotent stem cells undergo during the embryonic and fetal stage to such an extent that the mature cells are indistinguishable from native cells of the human body. Inherently, in an embodiment of the present invention, the cells are artificial.

As used herein, the term “artificial” in reference to cells may comprise material naturally occurring in nature but modified to a construct not naturally occurring. This includes human stem cells, which are differentiated into non-naturally occurring cells mimicking the cells of the human body.

### Protocol

Throughout this application the terms “method” and “protocol” when referring to processes for culturing or differentiating cells may be used interchangeably.

As used herein, the term “day” and similarly day in vitro (DIV) in reference to the protocols refers to a specific time for carrying out certain steps during the differentiation procedure.

In general and unless otherwise stated “day 0” refers to the initiation of the protocol, this is by for example but not limited to plating the stem cells or transferring the stem cells to an incubator or contacting the stem cells in their current cell culture medium with a compound prior to transfer of the stem cells. Typically, the initiation of the protocol will be by transferring undifferentiated stem cells to a different cell culture medium and/or container such as but not limited to by plating or incubating, and/or with the first contacting of the undifferentiated stem cells with a compound or compounds that affects the undifferentiated stem cells in such a way that a differentiation process is initiated.

When referring to “the cells” in a method is meant all cells of the cell population, regardless of cell type.

When referring to “day X”, such as day 1, day 2 etc., it is relative to the initiation of the protocol at day 0. One of ordinary skill in the art will recognize that unless otherwise specified the exact time of the day for carrying out the step may vary. Accordingly, “day X” is meant to encompass a time span such as of +/−10 hours, +/−8 hours, +/−6 hours, +/−4 hours, +/−2 hours, or +/−1 hours.

### Culturing Stem Cells

As used herein, the term “culturing” refers to a continuous procedure, which is employed throughout the method in order to maintain the viability of the cells at their various stages. After the cells of interest have been isolated from, for example but not limited to, living tissue or embryo, they are subsequently maintained under carefully controlled conditions. These conditions vary for each cell type, but generally consist of a suitable vessel with a substrate and/or medium that supplies the essential nutrients (amino acids, carbohydrates, vitamins, minerals), growth factors, hormones, and gases (CO₂, O₂), and regulates the physio-chemical environment (pH buffer, osmotic pressure, temperature).

As used herein, the term “cell culture medium” refers to a liquid or gel designed to support the growth of cells. Cell culture media generally comprise an appropriate source of energy and compounds which regulate the cell cycle.

As used herein, the term “incubator” refers to any suitable incubator that may support a cell culture. Non-limiting examples include culture dish, petri dish and plate (microtiter plate, microplate, deep well plate etc. of 6 well, 24 well, 48 well, 96 well, 384 well, 9600 well and the like), flask, chamber slide, tube, Cell Factory, roller bottle, spinner flask, hollow fiber, microcarrier, or bead.

As used herein, the term “providing stem cells” when referred to in a protocol means obtaining a batch of cells by methods such as described above and optionally transferring the cells into a different environment such as by seeding onto a new substrate. One of ordinary skill in the art will readily recognize that stem cells are fragile to such transfer and the procedure requires diligence and that maintaining the stem cells in the origin cell culture medium may facilitate a more sustainable transfer of the cells before replacing a cell culture medium with another cell culture medium more suitable for a further differentiation process.

### Differentiating Stem Cells

As used herein, the term “expressing” in relation to a gene or protein refers to the presence of an RNA molecule, which can be detected using assays such as reverse transcription quantitative polymerase chain reaction (RT-qPCR), RNA sequencing and the like, and/or a protein, which can be detected for example using antibody-based assays such as flow cytometry, immunocytochemistry/immunofluorescence, and the like. Depending on the sensitivity and specificity of the assay, a gene or protein may be considered expressed when a minimum of one molecule is detected such as in RNA sequencing, or the limit of detection above background/noise levels may be defined in relation to control samples such as in flow cytometry. A person skilled in the art will readily understand that when referring to the “expression” of a cell population, such as “the cell population expressing X % of a marker Y”, is meant that X % of cells in said cell population express the marker Y.

As used herein, the term “co-expression” means than an individual cell expresses a number of markers.

As used herein, the term “marker” refers to a naturally occurring identifiable expression made by a cell, which can be correlated with certain properties of the cell. In a preferred embodiment the marker is a genetic or proteomic expression, which can be detected and correlated with the identity of the cell. The markers may be referred to by gene. This can readily be translated into the expression of the corresponding mRNA and proteins.

As used herein, the term “negative” or “−” when used in reference to any marker such as a surface protein or transcription factor disclosed herein refers to the marker not being expressed in a cell or a population of cells, while the term “weak” or “low” refers to the marker being expressed at a reduced level in a cell as compared to the mean expression of the marker in a population of cells or as compared to a reference sample.

As used herein, the term “positive” or “+” when used in reference to any marker such as a surface protein or transcription factor disclosed herein refers to the marker being expressed in a cell or a population of cells, while the term “high” or “strong” refers to the marker being expressed at an increased level in a cell as compared to the mean expression of the marker in a population of cells or as compared to a reference sample.

As used herein, the term “differentiation” refers broadly to the process wherein cells progress from an undifferentiated state or a state different from the intended differentiated state to a specific differentiated state, e.g., from an immature state to a less immature state or from an immature state to a mature state, which may occur continuously as the method is performed. The term “differentiation” in respect to pluripotent stem cells refers to the process wherein cells progress from an undifferentiated state to a specific differentiated state, i.e., from an immature state to a less immature state or to a terminal state. Changes in cell interaction and maturation occur as cells lose markers of undifferentiated cells or gain markers of differentiated cells. Loss or gain of a single marker can indicate that a cell has “fully differentiated” or “terminally differentiated”. “Terminally differentiated” cells are the final stage of a developmental lineage and cannot further differentiate.

As used herein, by the term “contacting” in reference to culturing or differentiating cells is meant exposing the cells to e.g., a specific compound by placing the specific compound in a location that will allow it to touch the cell in order to produce “contacted” cells. The contacting may be accomplished using any suitable means. A non-limiting example of contacting is by adding the compound to a cell culture medium of the cells. The contacting of the cells is assumed to occur as long as the cells and specific compound are in proximity, e.g., the compound is present in a suitable concentration in the cell culture medium.

As used herein, the term “inhibitor” refers to a compound that reduces or suppresses or down-regulates a process, such as a signaling pathway which can promote cell differentiation.

As used herein, the term “activator” refers to a compound that induces or stimulates or up-regulates a process, such as a signaling pathway which can promote cell differentiation.

### Stem Cell-Derived Products

As used herein, the term “differentiated cells” refers to cells such as pluripotent stem cells which have progressed from an undifferentiated state to a less immature state. Differentiated cells may be e.g., less immature specialized cell such as progenitor cells or matured fully into a specialized/terminal cell type.

As used herein, the term “cell population” refers to a plurality of cells in the same culture. The cell population may be e.g., a mixture of cells of different types or cells at various developmental stages such as cells at various maturity stages towards the same or similar specialized feature or it may be a more homogeneous composition of cells with common markers.

As used herein, the term “neural cell population” refers to a cell population comprising neural cells.

As used herein, the terms “genetically modified” and “genetically engineered” in reference to a cell may be used interchangeably and refer to a cell which has been subjected to an artificial manipulation, modification, or recombination of DNA or other nucleic acid molecules in order to alter the characteristics (phenotype) of that cell. Such a cell can no longer be considered a naturally occurring cell. In case of genetically modified stem cells the traits resulting from the gene editing persist even as the stem cell is further differentiated into a specialized cell, thus rendering the specialized cell genetically modified and artificial, i.e., non-naturally occurring. An example of genetically modified stem cells are HLA-deficient stem cells, which are also referred to as universal donor cells and intended to overcome the problem of graft rejection. A method for obtaining HLA-deficient stem cells is disclosed in WO/2020/260563.

### Neuroectodermal Cells

As used herein, the term “neural” refers to the nervous system.

As used herein, the term “neural cell” (unless otherwise stated) refers to a cell, where the native counterpart naturally forms part of the ectoderm germ layer, more specifically the neuroectoderm and is meant to encompass cells at any stage of development within this germ layer, such as NSCs all the way through to neurons and other terminally differentiated cell types (e.g., glial cells), i.e., cell stages such as neural stem cell stage and neuroblast stage. Accordingly, neurons and precursors thereof are considered specific types of neural cells.

As used herein, the terms “neuron” and “nerve cell” may be used interchangeably referring to neural cells which are post-mitotic and have terminally differentiated into a specialized cell. A neuron is characterized by expression of the marker INA, or other equivalent markers such as ELAVL3, ELAVL4 (typically detected with the antibody HuC/D), RBFOX3 (typically detected with the antibody NeuN), STMN2, NCMA1 or other such broad neuronal markers.

As used herein, the term “neural stem cell” or “NSC” and “neural precursor cell” or “NPC” are terms that are used interchangeably to refer to a self-renewing, multipotent cell of the nervous system capable of giving rise to a vast array of more specialized cells of the CNS and PNS. NSCs and NPCs typically expressing transcription factors such as SOX2, NES, PAX6, SOX1, OTX2, OTX1, NKX6.1, OLIG2, NKX2.2, FOXG1, FOXA2 or LMX1A.

As used herein, the term “neuroblast cell” refers to an intermediate precursor cell, which is typically multipotent or only unipotent and can self-renew only to a limited extent. A neuroblast cell finally gives rise to terminally differentiated cell types such as neurons. The terms “neuroblast cell” and “intermediate precursor cell” and “intermediate progenitor cell” and “radial glial cell” may be used interchangeably.

As used herein, the term “neuroblast” or “intermediate precursor cell” means a cell that has expressed a gene associated with this stage such as ASCL1, SOX4, MASH1, EOMES, NHLH1, any of the NEUROD or NEUROG gene family or other such genes. These cells are those which are destined to become neurons.

As used herein, the terms “neuron progenitor”, “precursor of a neuron” and “neuron precursor” may be used interchangeably and refer to a neural cell with the potential or propensity to further specialize into a neuron. The terms “neuron precursor” and “non-native neuron precursor” may be used interchangeably.

The neural cells according to the present invention may have a specific regional identity, such as cells specific to the midbrain.

As used herein, the term “forebrain” refers to the rostral region of the neural tube and CNS that gives rise to structures including the cerebral cortex and the striatum.

As used herein, the term “midbrain” refers to the medial region of the neural tube and CNS (on the rostro-caudal axis) that gives rise to structures including the substantia nigra.

As used herein, the term “ventral midbrain” in reference to a cell means a neural cell having certain properties of a neural cell naturally occurring in the ventral midbrain. Typically, ventral midbrain neural cells are characterized by the expression of certain markers such as FOXA2 and LMX1A. Specifically, as used herein the term “ventral midbrain neural stem cell” refers to a neural stem cell having the characteristics of a neural stem cell naturally occurring in the ventral midbrain. A ventral midbrain NSC or NPC may be characterized by the co-expression of markers FOXA2, LMX1A, EN1, OTX2, and SOX2.

As used herein, the terms “hindbrain” and “spinal cord” refer to the caudal regions of the neural tube that are caudal to the isthmus organizer.

As used herein, the term “dopaminergic (DA) cell” or “dopaminergic neuron” or “dopamine neuron” refers to a cell that is capable of synthesizing the neurotransmitter dopamine.

As used herein, the term “stromal cell” refers to cells having the capacity to become connective tissue cells or cells of a fibroblast identity.

As used herein, the term “VLMC” means vascular leptomeningeal cells and is consider a type of stromal cell resident in the CNS.

As used herein, the term “glial cell” refers to cells that are non-neuronal cells in the central nervous system (brain and spinal cord) and the peripheral nervous system that do not produce electrical impulses, that perform support and protection for neurons. Examples include astrocytes and oligodendrocytes and their precursors, glial precursor cells or glioblasts.

Directing Differentiation into Ventral Midbrain Neurons

In a general aspect of the present invention is provided a method comprising contacting a cell population comprising ventral midbrain NSCs with an inhibitor of MEK signaling and an inhibitor of NOTCH signaling. Specifically, in an embodiment the method is for directing differentiation of ventral midbrain NSCs into neurons. In a further embodiment, the method is for differentiating ventral midbrain NSCs into ventral midbrain neurons. In a preferred embodiment, the ventral midbrain NSCs are neurally induced, ventralized, and caudalized. In an embodiment, the cell population is neurally induced, ventralized, and caudalized to obtain ventral midbrain NSCs. In an embodiment, the method is in vitro.

The method is intended to increase the number of cells in the cell population having a neuronal fate. As used herein, the term “neuronal fate” in reference to a cell means the developmental fate of that cell becomes restricted such that it will develop into a neuron.

As used herein, the term “directing differentiation” means affecting the developmental fate of a cell towards a certain outcome. Directing the differentiation of a cell is not necessarily a continuous process and the cell may not necessarily develop into the final fate during the method as disclosed herein. In a preferred embodiment, the differentiation of the cells is directed in vitro so that the cells may later develop in vivo towards a certain fate, specifically a neuronal fate.

It follows that in a general embodiment, the method is for increasing the proportion of neural cells differentiating into neurons. In an embodiment, the method is for decreasing the proportion of neural cells differentiating into stromal cells, such as vascular leptomeningeal cells (VLMCs), non-vmDA neurons, and/or astrocytes.

As used herein, the term “MEK” refers to MAPK/ERK kinase and the term “MEK signaling” refers to the activation of MAPK/ERK pathway (also known as the Ras-Raf-MEK-ERK pathway). As used herein, the term “inhibitor of MEK signaling” (MEKi) refers to any compound that inactivates the MAPK/ERK pathway. In an embodiment, the inhibitor of MEK signaling inhibits MKK1 (MEK1) and MKK2 (MEK2). In an embodiment, the inhibitor of MEK signaling inhibits the activation and downstream signaling of MEK. In an embodiment, the inhibitor of MEK signaling is a potent inhibitor suppressing the phosphorylation of ERK. This applies to all aspects of the present invention.

As used herein, the term “NOTCH” refers to the signaling pathway of the notch receptors. As used herein, the term “inhibitor of NOTCH signaling” (NOTCHi) refers to any compound that inactivates the NOTCH pathway. In an embodiment, the inhibitor of NOTCH signaling does so by targeting the gamma-secretase or γ-secretase complex. This applies to all aspects of the present invention. The present inventors have further found that for directing a cell population towards a ventral midbrain neuronal fate contacting the cell population with an inhibitor of MEK signaling and an inhibitor of NOTCH signaling provides a synergistic effect.

In an embodiment, at least 5%, such as at least 10%, 15%, 20%, 25%, 30%, 35%, 40%, 45%, 50%, 55%, 60%, 65%, 70%, 75%, 80%, 85%, or 90% of the cell population comprising ventral midbrain NSCs co-express the markers FOXA2, LMX1A, EN1, OTX2, and SOX2. The present inventors have found that the effect in increasing neuronal differentiation by the inhibition of MEK signaling is particular pronounced on NSCs that are neurally induced, ventralized and caudalized to such an extent that they co-express the markers FOXA2, LMX1A, EN1, OTX2, and SOX2.

The present inventors have identified an expression profile of the cell population comprising ventral midbrain NSCs that provides an improved outcome of neurons. In an embodiment, 0.5-65%, 5-65%, 10-65%, 15-65%, 20-65%, 25-65%, 30-65%, 35-65%, or 40-65%, preferably 30-65%, more preferably 40-65%, of the cell population comprising ventral midbrain NSCs express ASCL1 at the time of contacting the cell population with the inhibitor of MEK signaling and the inhibitor of NOTCH signaling. In an embodiment, 25-65%, 30-65%, 35-65, 40-65%, or 45-65%, preferably 45-65%, of the cell population comprising ventral midbrain NSCs express KI67 at the time of contacting the cell population with the inhibitor of MEK signaling and the inhibitor of NOTCH signaling. In an embodiment, less than 20%, preferably less than 15%, of the cell population comprising ventral midbrain NSCs express INA at the time of contacting the cell population with the inhibitor of MEK signaling and the inhibitor of NOTCH signaling. In an embodiment, 0-20%, 0.1-20%, 0-15%, 0.1-15%, 5-20%, 10-20%, 5-15%, or 10-15%, preferably 10-15%, of the cell population comprising ventral midbrain NSCs express INA at the time of contacting the cell population with the inhibitor of MEK signaling and the inhibitor of NOTCH signaling. In an embodiment, less than 10%, preferably less than 5%, of the cell population comprising ventral midbrain NSCs are INA+/SOX2− at the time of contacting the cell population with the inhibitor of MEK signaling and the inhibitor of NOTCH signaling. In an embodiment, 0-10%, 0-5%, 1-5%, or 2-5%, preferably 2-5%, of the cell population comprising ventral midbrain NSCs are INA+/SOX2− at the time of contacting the cell population with the inhibitor of MEK signaling and the inhibitor of NOTCH signaling. In an embodiment, 75-95%, preferably 80-95% of the cell population comprising ventral midbrain NSCs express SOX2 at the time of contacting the cell population with the inhibitor of MEK signaling and the inhibitor of NOTCH signaling. In a preferred embodiment, all of the above applies, i.e., 40-60% of the cell population comprising ventral midbrain NSCs express ASCL1, 45-65% of the cell population comprising ventral midbrain NSCs express KI67, 10-15% of the cell population comprising ventral midbrain NSCs express INA, 2-5% of the cell population comprising ventral midbrain NSCs are INA+/SOX2−, and 80-95% of the cell population comprising ventral midbrain NSCs express SOX2 at the time of contacting the cell population with the inhibitor of MEK signaling and the inhibitor of NOTCH signaling. In a preferred embodiment, the cell population is not contacted with an inhibitor of NOTCH signaling, such as DAPT, prior to having the aforementioned preferred expression profile.

In an embodiment, the cell population comprising ventral midbrain NSCs is contacted with the inhibitor of MEK signaling and the inhibitor of NOTCH signaling prior to more than 70%, or 60%, of the cell population having further developed into intermediate neural precursors. In another embodiment, the cell population comprising ventral midbrain NSCs is contacted with the inhibitor of MEK signaling and the inhibitor of NOTCH signaling prior to more than 20%, 15%, 10%, or 5%, of the cell population having further developed into intermediate neural precursors or neurons. Specifically, in another embodiment, the cell population comprising ventral midbrain NSCs is contacted with the inhibitor of MEK signaling and the inhibitor of NOTCH signaling, prior to more than 20%, 15%, 10%, or 5%, of the cell population express one of the markers ASCL1 and INA.

As used herein, the term “intermediate neural precursor” refers to a cell at a developmental stage between neural stem cell and neuron. A ventral midbrain neural cell at this stage is characterized by the expression of the marker ASCL1. The present inventors have found that the window of opportunity for efficiently directing the development of the cell population into neurons is prior to the cells maturing to the stage where they become neurons or other terminal neural cells such as astrocytes, stromal cells, such as VLMCs etc.

In an embodiment, the inhibitor of MEK signaling is selected from PD0325901, trametinib (GSK1120212), selumetinib (AZD6244), pimasertib (AS703026), MEK162, cobimetinib, PD184352, PD173074, BIX02189, AZD8330, PD318088, Refametinib, and PD98059. As used herein, PD0325901 refers to a small molecule with the chemical name N-[(2R)-2,3-Dihydroxypropoxy]-3,4-difluoro-2-[(2-fluoro-4-iodophenyl)amino]-benzamide with CAS no. 391210-10-9, and is a potent MKK1 (MEK1) and MKK2 (MEK2) inhibitor.

In an embodiment, the concentration of the inhibitor of MEK signaling is at least 5 μM, preferably at least 10 μM. In an embodiment, the concentration of the inhibitor of MEK signaling is from 5 μM to 100 μM, from 5 μM to 50 μM, from 5 μM to 40 μM, or from 5 μM to 30 μM.

In an embodiment, the inhibitor of NOTCH signaling is selected from DAPT, Avagacestat, PF-03084014, and LY450139. As used herein, the term “DAPT” refers to a small molecule with the chemical name (2S)—N-[(3,5-Difluorophenyl)acetyl]-L-alanyl-2-phenyl]glycine 1,1-dimethylethyl ester with CAS no. 208255-80-5. As used herein, the term “Avagacestat” refers to a compound having CAS No. 1146699-66-2. As used herein, the term “PF-03084014” refers to a compound having CAS No. 1290543-63-3. As used herein, the terms “LY450139” and “Semagacestat” may be used interchangeably and refer to a compound having CAS No. 425386-60-3.

In an embodiment, the concentration of the inhibitor of NOTCH signaling is at least 1 μM, preferably at least 10 μM. In a further embodiment, the concentration of the inhibitor of NOTCH signaling is from 1 μM to 100 μM, 1 μM to 50 μM, from 1 μM to 40 μM, or from 1 μM to 30 μM.

In an embodiment, the cell population is contacted with the inhibitor of MEK signaling for at least % hour, 1 hour, 2, 3, 4, 5, 6, 7, 8, 9, 10, 11, or 12 hours, or 18 hours, or 24 hours, or for at least 2 days, 3 days, 4 days, 5 days, or 6 days. In an embodiment, the cell population is contacted with the inhibitor of MEK signaling for % hour to 15 days, 2 hour to 10 days, % hour to 5 days, % hour to 2 days, 1 hour to 2 days, 3 hours to 2 days, 6 hours to 2 days, 12 hours to 2 days, 18 hours to 2 days, or 1 to 2 day(s), or for about 2 to about 15 days. In an embodiment, the cell population is contacted with the inhibitor of NOTCH signaling for at least % hour, 1 hour, 2, 3, 4, 5, 6, 7, 8, 9, 10, 11, or 12 hours, or 18 hours, or 24 hours, or for at least 2 days, 3 days, 4 days, 5 days, or 6 days. In an embodiment, the cell population is contacted with the inhibitor of NOTCH signaling for % hour to 15 days, 2 hour to 10 days, % hour to 5 days, % hour to 2 days, 1 hour to 2 days, 3 hours to 2 days, 6 hours to 2 days, 12 hours to 2 days, 18 hours to 2 days, or 1 to 2 day(s), or for about 2 to about 15 days.

In an embodiment, the inhibitor of MEK signaling and the inhibitor of NOTCH signaling is administered to the cells at least every 48 hours, 36 hours, preferably at least every 30 hours, more preferably at least every 24 hours. In an embodiment, the inhibitor of MEK signaling and the inhibitor of NOTCH signaling is replaced at least every 12 to 36 hours, preferably at least every 18 to 30 hours, more preferably at least every 24 hours. In a preferred embodiment, the cell population during the time of contacting with the inhibitors it is ensured that the cells are sufficiently exposed to the inhibitors. In an embodiment, the cell population is continually exposed through sufficient media replacement, such as every 24 hours. Alternatively, the cell population may be sufficiently exposed to the inhibitors via a biomaterial or substance that ensures continued release of the inhibitors.

In an embodiment, the cell population is contacted with the inhibitor of MEK signaling and the inhibitor of NOTCH signaling at least partly at the same time. In an embodiment, the cell population is contacted with the inhibitor of MEK signaling and the inhibitor of NOTCH signaling simultaneously.

According to the present invention, the cells are directed towards differentiation into neurons in vitro, in order to be suitable for later administration into a patient wherein the further development in vivo results in a neuronal fate. Administration into the patient is likely not occur immediately following the method for directing differentiation towards neuronal development. Accordingly, in an embodiment, the cell population is cryopreserved following the inhibition of MEK and NOTCH signaling. In an embodiment, the cell population is cryopreserved within 4 days, such as within 3 days, 2 days, 1 day, 12 hours, 6 hours, or 3 hours, following end of inhibition of MEK and NOTCH signaling. In an embodiment, the cell population is cryopreserved immediately following end of inhibition of MEK and NOTCH signaling. In an embodiment, the cell population is cryopreserved in DMSO, or using a cryoprotectant not containing DMSO. A person skilled in the art will know that such cryoprotectants are commercially available and techniques for cryopreserving a cell population, such as one comprising neural cells, are well-known.

According to the present invention contacting the cell population comprising ventral midbrain NSCs with an inhibitor of MEK signaling and an inhibitor of NOTCH signaling directs a cell population towards a ventral midbrain neuronal fate. Accordingly, the cell population is contacted with one or both of the inhibitors when at least part of the cells have differentiated into ventral midbrain NSCs. Notwithstanding, the cells may be contacted with one or both of the inhibitors prior to this stage to achieve similar or other effects. However, in a preferred embodiment, the cell population is not contacted with an inhibitor of MEK signaling or an inhibitor of NOTCH signaling prior to the cell population having been neurally induced, ventralized, and caudalized. In a preferred embodiment, the cell population is not contacted with an inhibitor of NOTCH signaling, such as DAPT, prior to the contacting of the cell population with the inhibitor of MEK signaling and the inhibitor of NOTCH signaling according to the method.

The present inventors identified an expression profile of a cell population comprising NSCs as particularly suitable for directing differentiation towards neurons. Accordingly, an aspect of the present invention also relates to an in vitro method for directing differentiation of ventral midbrain NSCs into neurons comprising contacting a cell population comprising ventral midbrain NSCs with an inhibitor of MEK signaling and an inhibitor of NOTCH signaling, wherein 0.5-66%, preferably 40-60%, of the cell population comprising ventral midbrain NSCs express ASCL1, 25-65%, preferably 45-65%, of the cell population comprising ventral midbrain NSCs express KI67, 0-20%, preferably 10-15%, of the cell population comprising ventral midbrain NSCs express INA, 0-10%, preferably 2-5%, of the cell population comprising ventral midbrain NSCs are INA+/SOX2−, and 75-95%, preferably 80-95%, of the cell population comprising ventral midbrain NSCs express SOX2. A person skilled in the art will appreciate that the embodiments referred to above equally apply to this specific aspect.

Differentiation of PSCs into Ventral Midbrain NSCs

The general aspect relating to the method for directing differentiation of ventral midbrain NSCs into neurons requires as a starting material the cell population comprising the ventral midbrain NSCs. The cell population comprising ventral midbrain NSCs may be obtained by any suitable method. Accordingly, in an embodiment, the method comprises an initial step of differentiating a cell population into ventral midbrain NSCs. In an embodiment, the cell population is derived from PSCs. In such an embodiment, the cell population is neurally induced, ventralized, and caudalized to obtain ventral midbrain NSCs. Accordingly, in an embodiment, prior to contacting the cell population with the inhibitor of MEK signaling and an inhibitor of NOTCH signaling, the cell population is cultured to induce differentiation into ventral midbrain NSCs.

In an embodiment, prior to contacting the cell population with the inhibitor of MEK signaling and the inhibitor of NOTCH signaling, the cell population is contacted with an inhibitor of Small Mothers Against Decapentaplegic (SMAD) protein signaling, an activator of Sonic Hedgehog (SHH) signaling, an activator of wingless (Wnt) signaling, and/or an activator of fibroblast growth factor (FGF) signaling, and, optionally, ascorbic acid and/or, optionally, Brain-derived neurotrophic factor (BDNF).

In an embodiment for differentiating PSCs into ventral midbrain NSCs, the cell population is differentiated into ventral midbrain neural cells by neurally inducing the cell population by contacting the cell population with an inhibitor of SMAD protein signaling, such as at least two inhibitors of SMAD protein signaling. As used herein, the term “SMAD protein signaling” refers to the Small Mothers Against Decapentaplegic (SMAD) protein signaling pathway. A person skilled in the art will recognize that contacting PSCs with one or more inhibitors of the SMAD signaling pathway is a robust technique for differentiating cells into the neural lineage. In an embodiment, the inhibitor of the SMAD signaling pathway is selected from Noggin, LY364947, SB431542, RepSox, and LDN-193189, or a similar compound.

In an embodiment for differentiating PSCs into ventral midbrain NSCs, the cell population is differentiated into ventral midbrain neural stem cells by inducing ventralization of the cell population. As used herein, the terms “ventralization” and “ventral patterning” may be used interchangeably and refer to the process whereby pluripotent cells assume a ventral gene expression identity equivalent to cells of an embryo or embryonic structure, i.e., neural tube. In an embodiment, the PSCs are ventralized by contacting the cells with an activator of the SHH signaling pathway. As used herein, the term “activator of sonic hedgehog signaling” refers to any molecule or compound that is capable of activating a SHH signaling pathway. Activation of the SHH pathway is well known as being responsible for the induction and maintenance of ventral neural tube structures. In an embodiment, the activator of SHH signaling is selected from SHH, purmorphamine, and SAG, or a similar compound.

In an embodiment for differentiating PSCs into ventral midbrain NSCs, the cell population is differentiated into ventral midbrain neural cells by inducing caudalization of the cell population. As used herein, the term “caudalization” refers to the process whereby pluripotent cells assume a caudal gene expression identity equivalent to cells of an embryo or embryonic structure, i.e., neural tube. In an embodiment, the PSCs are caudalized by contacting the cells with an activator of the Wnt signaling pathway. As used herein, the term “activator of Wnt signaling” refers to any molecule or compound that is capable of activating a Wnt signaling pathway. Inhibitors of Wnt signaling is well known as being responsible for caudalizing of neural cells. In an embodiment, the activator of Wnt signaling lowers GSK3-beta for activation of Wnt signaling. Accordingly, in certain embodiments, the Wnt activator is an inhibitor of GSK3-beta. In an embodiment, the activator of Wnt signaling is selected from CHIR99021 and a recombinant Wnt protein.

In an embodiment for differentiating PSCs into ventral midbrain NSCs, the cell population is differentiated into ventral midbrain neural cells by further inducing caudalization of the cell population. Accordingly, in an embodiment, the cell population is contacted with an activator of fibroblast growth factor (FGF). In an embodiment, the activator of FGF signaling is FGF8b.

In a certain embodiment, the concentration of the inhibitor(s) of SMAD protein signaling is from 1 μM to 50 μM, the activator of SHH signaling is from 200 ng/ml to 800 ng/ml, the inhibitor of Wnt signaling is from 0.1 μM to 1 μM, the activator of FGF signaling is from 10 ng/ml to 200 ng/ml, ascorbic acid is from 50 μM to 500 μM, and/or BDNF is from 1 ng/ml to 50 ng/ml.

In a preferred embodiment, the PSCs are human embryonic stem cells or human induced pluripotent stem cells.

A further aspect of the present invention relates to an in vitro method for directing differentiation of a cell population of PSCs into ventral midbrain neurons, comprising culturing the cell population of PSCs, contacting the cell population of PSCs with an inhibitor of SMAD protein signaling, an inhibitor of Wnt signaling, an activator of SHH signaling, an activator of FGF signaling, optionally ascorbic acid, and, optionally BDNF, to obtain a cell population comprising ventral midbrain NSCs, wherein the cell population comprising ventral midbrain NSCs is further contacted with an inhibitor of MEK signaling and an inhibitor of NOTCH signaling to direct differentiation into ventral midbrain neurons.

In an embodiment, the cell population of pluripotent stem cells is contacted with the inhibitor of SMAD protein signaling for 5 to 9 days. In a certain embodiment, the cell population is contacted with the inhibitor of SMAD protein signaling for 5 to 9 days from day 0.

In an embodiment, the cell population of PSCs is contacted with the activator of SHH signaling for 5 to 9 days, such as 7 to 9 days, preferably for 9 days. In a preferred embodiment, the cell population is contacted with the inhibitor of Wnt signaling for 5 to 9 days from day 0. In an embodiment, the concentration of the activator of SHH signaling is from 200 ng/ml to 800 ng/ml.

In an embodiment, the cell population of pluripotent stem cells is contacted with the inhibitor of Wnt signaling for 5 to 9 days, such as 7 to 9 days, preferably for 9 days. In a preferred embodiment, the cell population is contacted with the inhibitor of Wnt signaling for 5 to 9 days from day 0. In an embodiment, the concentration of the activator of Wnt signaling is from 0.1 μM to 1 μM.

In an embodiment, the cell population of PSCs is contacted with the activator of FGF signaling for 7 to 12 days following ended contacting with the inhibitor of SMAD protein signaling, inhibitor of Wnt signaling, and/or activator of SHH signaling. In another embodiment, the cell population is contacted with the activator of FGF signaling for 7 to 12 days from day 5 to 9, or when ending contacting with the inhibitor of SMAD protein signaling, inhibitor of Wnt signaling, and/or activator of SHH signaling. In a certain embodiment, the cell population is contacted with the inhibitor of SMAD protein signaling, the activator of SHH signaling, and the inhibitor of Wnt signaling from day 0 to day 9, subsequently, the cell population is contacted with the activator of FGF signaling from day 9 to day 16. In an embodiment, the activator of FGF signaling is FGF8b. In an embodiment, the concentration of the activator of FGF signaling is from 10 ng/ml to 200 ng/ml.

In an embodiment, the cell population is contacted with ascorbic acid for 5 to 7 days from day 10 or 11. In an embodiment, the concentration of ascorbic acid is from 10 μM to 400 μM, preferably from 100 μM to 300 μM, preferably from 150 μM to 250 μM, more preferably about 200 μM.

In an embodiment, the cell population is contacted with BDNF for 5 to 7 days from day 10 or 11. In an embodiment, the concentration of BDNF is from 1 ng/ml to 40 ng/ml, preferably from 10 ng/ml to 40 ng/ml, preferably 15 ng/ml to 30 ng/ml, more preferably about 20 ng/ml.

In an embodiment, the cell population is allowed to differentiate into ventral midbrain NSCs for 14 to 24 days, such as for 15 to 20 days, prior to contacting the cell population with the inhibitor of MEK signaling and the inhibitor of NOTCH signaling. In an embodiment, the cell population is contacted with the inhibitor of MEK signaling and the inhibitor of NOTCH signaling for % hour to 15 days, % hour to 10 days, % hour to 5 days, % hour to 2 days, 1 hour to 2 days, 3 hours to 2 days, 6 hours to 2 days, 12 hours to 2 days, 18 hours to 2 days, or 1 to 2 day(s), or for about 2 to about 10 days. In an embodiment, the cell population is contacted with the inhibitor of MEK and NOTCH from 0 to 10 days after the cell population is no longer contacted with the activator of FGF signaling.

In a preferred embodiment, while the cell population is allowed to differentiate into ventral midbrain NSCs, the cell population is not contacted with an inhibitor of NOTCH signaling, such as DAPT.

In an embodiment, prior to contacting the cell population with the inhibitor of MEK signaling and the inhibitor of NOTCH signaling, the cell population comprising ventral midbrain NSCs is cultured for 6 to 8 days from day 16 in a suitable culture medium without contacting the cell population with an inhibitor of SMAD protein signaling, an inhibitor of Wnt signaling, an activator of SHH signaling, an activator of FGF signaling, or an inhibitor of NOTCH signaling, such as DAPT. A person skilled in the art will recognize that while the cell population is further cultured without such components the cells will continue to differentiate.

In an alternative embodiment, the cell population is contacted with an inhibitor of Wnt signaling for at least 14 days instead of contacting the cell population with an activator of FGF. In such an embodiment, the cells are then contacted with the inhibitor of MEK and NOTCH from 0 to 10 days after the cell population is no longer contacted with the inhibitor of Wnt signaling.

In an embodiment the cell population is harvested before 28 days from beginning differentiation of the cell population into ventral midbrain NSCs, such as before 27 days, 26 days, or 25 days, preferably before 26 days.

In an embodiment, the cell population is harvested at day 28, day 27, day 26, or day 25 from beginning differentiation of the cell population into ventral midbrain NSCs, preferably at day 25.

As used herein, the term “harvested” means that the cells are collected and transferred to a new environment. This may be one wherein the cells are re-seeded into a new in vitro culture system. This may also be one where cells do not further develop into neurons as in an embodiment, the cell population is cryopreserved at harvest. Accordingly, in an embodiment, the cell population is cryopreserved following the inhibition of MEK signaling and inhibition of NOTCH signaling. In an embodiment, the cell population is cryopreserved within 4 days, such as within 3 days, 2 days, 1 day, 12 hours, 6 hours, 3 hours, or 1 hour following end of inhibition of MEK signaling and inhibition of NOTCH signaling. In an embodiment, the cell population is cryopreserved within 4 days, such as within 3 days, 2 days, 1 day, 12 hours, 6 hours, or 3 hours, following harvest.

In an embodiment, the cell population is contacted with the inhibitor of MEK signaling and the inhibitor of NOTCH signaling at least partly at the same time. In a certain embodiment, the cell population is contacted with the inhibitor of MEK signaling and the inhibitor of NOTCH signaling simultaneously.

In an embodiment, the cell population is cultured in a two-dimensional culture. In a further embodiment, the cell population is initially plated on a substrate. In an embodiment, the substrate comprises an extracellular matrix. In a further embodiment, the substrate comprises Poly-L-Lysine, Poly-D-Lysine, Poly-Ornithine, laminin, fibronectin, and/or collagen, and/or fragments thereof. In a more specific embodiment, the laminin or fragment thereof is selected from the group comprising of laminin-111, laminin-521, and laminin-511. In an embodiment, the cell population is cultured on a laminin-111 substrate. In an embodiment, the concentration of the laminin substrate is about 10 μg/ml.

In an embodiment, 0.5-65%, 5-65%, 10-65%, 15-65%, 20-65%, 25-65%, 30-65%, 35-65%, or 40-65%, preferably 30-65%, more preferably 40-65%, of the cell population comprising ventral midbrain NSCs express ASCL1 at the time of contacting the cell population with the inhibitor of MEK signaling and the inhibitor of NOTCH signaling. In an embodiment, 25-65%, 30-65%, 35-65, 40-65%, or 45-65%, preferably 45-65%, of the cell population comprising ventral midbrain NSCs express KI67 at the time of contacting the cell population with the inhibitor of MEK signaling and the inhibitor of NOTCH signaling. In an embodiment, less than 20%, preferably less than 15%, of the cell population comprising ventral midbrain NSCs express INA at the time of contacting the cell population with the inhibitor of MEK signaling and the inhibitor of NOTCH signaling. In an embodiment, 0-20%, 0.1-20%, 0-15%, 0.1-15%, 5-20%, 10-20%, 5-15%, or 10-15%, preferably 10-15%, of the cell population comprising ventral midbrain NSCs express INA at the time of contacting the cell population with the inhibitor of MEK signaling and the inhibitor of NOTCH signaling. In an embodiment, less than 10%, preferably less than 5%, of the cell population comprising ventral midbrain NSCs are INA+/SOX2− at the time of contacting the cell population with the inhibitor of MEK signaling and the inhibitor of NOTCH signaling. In an embodiment, 0-10%, 0-5%, 1-5%, or 2-5%, preferably 2-5%, of the cell population comprising ventral midbrain NSCs are INA+/SOX2− at the time of contacting the cell population with the inhibitor of MEK signaling and the inhibitor of NOTCH signaling. In an embodiment, 75-95%, preferably 80-95% of the cell population comprising ventral midbrain NSCs express SOX2 at the time of contacting the cell population with the inhibitor of MEK signaling and the inhibitor of NOTCH signaling. In a preferred embodiment, all of the above applies, i.e., 40-60% of the cell population comprising ventral midbrain NSCs express ASCL1, 45-65% of the cell population comprising ventral midbrain NSCs express KI67, 10-15% of the cell population comprising ventral midbrain NSCs express INA, 2-5% of the cell population comprising ventral midbrain NSCs are INA+/SOX2−, and 80-95% of the cell population comprising ventral midbrain NSCs express SOX2 at the time of contacting the cell population with the inhibitor of MEK signaling and the inhibitor of NOTCH signaling.

In an embodiment, the cell population is cultured by obtaining a neural microsphere, comprising the steps of obtaining neural stem precursor blast cells, aggregating the neural stem precursor blast cells to form a neural microsphere, and allowing the neural stem precursor blast cells of the neural microsphere to further mature. In an embodiment, the method comprises the additional step of seeding the neural stem precursor blast cells in a well suitable for maintaining a neural microsphere in a static non-adherent culture, prior to the step of aggregating the neural stem precursor blast cells. Methods for culturing the cell population in a neural microsphere is detailed in WO/2021/099532, hereby incorporated by reference.

### Late Neural Differentiation by Using an Inhibitor of NOTCH Signaling

Another aspect of the present invention relates to an in vitro method for directing differentiation of a cell population into ventral midbrain neurons, comprising the steps of culturing a cell population comprising PSCs, inducing differentiation of the cell population into ventral midbrain NSCs, allowing the ventral midbrain NSCs to mature, and contacting the cell population comprising ventral midbrain NSCs with an inhibitor of NOTCH signaling, wherein the ventral midbrain NSCs express the markers FOXA2, LMX1A, EN1, OTX2, and SOX2.

In an embodiment, the cell population is contacted with the inhibitor of NOTCH signaling at least 20 days, preferably 21 days, more preferably 22 days, after initially inducing differentiation of the cell population into ventral midbrain NSCs.

In an embodiment, the ventral midbrain NSCs are allowed to mature for at least 4 days before contacting the cell population comprising ventral midbrain NSCs with the inhibitor of NOTCH signaling.

In an embodiment, the ventral midbrain NSCs are contacted with the inhibitor of NOTCH signaling no earlier than 4 days after at et least 70% of the cell population express the markers FOXA2, LMX1A, EN1, OTX2, and SOX2.

### Cell Population of Ventral Midbrain Neural Cells

An aspect of the present invention relates to a cell population comprising ventral midbrain neural cells obtainable by any of the methods described herein. In a certain embodiment, the cell population comprising ventral midbrain neural cells is obtained by the method according to any one of the previous embodiments. Notably, the present inventors have not been able to identify parameters which distinguish the cell population harvested immediately following the inhibition of MEK signaling and inhibition of NOTCH signaling as compared to a cell population which have not been subject to the method of directing differentiation towards neurons according to the present invention. However, it is evident that the cells are stimulated by the signaling and that the proportion of cells holding neuronal fate has been increased, which immediately becomes apparent once the cell population is allowed to culture further. Accordingly, an aspect of the present invention relates to a cell population comprising ventral midbrain NSCs, wherein the cell population when cultured in vitro for 5 days in a culture medium suitable for maintaining neural cells results in a cell population wherein at least 50% are neurons as determined by HuCD, INA or related markers and <15% of cells are proliferating as determined by KI67, MKI67 or other such proliferative markers (Example 5). In an embodiment, the expression of markers by the cells is measured using scRNAseq according to Example 7. In an embodiment, the expression of markers by the cells is measured according to immunocytochemistry (ICC) as described in Example 8. In an embodiment, the expression of markers by the cells is measured according to flow cytometry as described in Example 2. In an embodiment, the cell population is cultured according to Example 6.

Accordingly, an aspect of the present invention relates to a cell population comprising ventral midbrain NSCs, wherein the cell population when cultured in vitro for 5 days in a culture medium suitable for maintaining neural cells results in a cell population wherein at least 25% are HuCD+/SOX2− or HUCD or NEUN+/SOX2−. In an embodiment, the expression of markers by the cells is measured using scRNAseq according to Example 7. In an embodiment, the expression of markers by the cells is measured according to immunocytochemistry (ICC) as described in Example 8. In an embodiment, the expression of markers by the cells is measured according to flow cytometry as described in Example 2. In an embodiment, the cell population is cultured according to Example 6.

In an embodiment, the cell population is in vitro. In an embodiment, the ventral midbrain neural cells are non-native. In an embodiment, the ventral midbrain neural cells are artificial. In a preferred embodiment, the ventral midbrain neural cells are stem cell-derived. In a certain embodiment, the ventral midbrain neural cells are stem cell-derived from pluripotent stem cells. In a further embodiment, the ventral midbrain neural cells are stem cell-derived from human embryonic stem cells (hESCs) or human induced pluripotent stem cells (hiPSCs).

In an embodiment, the cells of the cell population are genetically modified. In an embodiment, the pluripotent stem cells are genetically modified, and the genetic modifications persist in the ventral midbrain neural cells obtained according to the any of the methods described herein. In certain embodiments the cells of the cell population are genetically modified to become hypoimmunogenic. As used herein, the terms “hypoimmunogenic” and “immune evasive” in reference to a cell may be used interchangeably and refer to properties of the cell making it less prone to immune rejection by a subject into which such cells are transplanted. Typically, specific surface markers are overexpressed or silenced. In an embodiment, the genetically modified cells have reduced expression of MHC-I and/or MHC-II. In an embodiment, the cells are genetically modified to express one or more tolerogenic factors or analogues thereof, such as HLA-E, HLA-G, CD46, CD47, CD55, CD59, and PD-L1. Examples and methods for genetically modifying cells to be immune evasive are described in WO2012145384, WO2013158292, WO2016142532, WO2016183041, WO2018132783, WO2018175390, WO2019161271, WO2020018615, WO2020018620, WO2020049535, WO2020168317, WO2021195426, WO2022012591, and WO2020260563. In some aspects, genome editing technologies (e.g., the CRISPR/Cas or TALEN systems) are used to modulate (e.g., reduce, eliminate and/or increase) the expression of specific genes.

In an embodiment, the genetic modifications for hypoimmunogenicity comprise reduced expression of MHC-I human leukocyte antigens relative to a wild-type stem cell, reduced expression of MHC-II human leukocyte antigens relative to a wild-type stem cell, and/or increased expression of a tolerogenic factor relative to wild-type stem cell. In an embodiment, the MHC-I human leukocyte antigens are HLA-A, HLA-B, and HLA-C. In an embodiment, the MHC-II human leukocyte antigens are HLA-DP, HLA-DQ, and HLA-DR. In an embodiment, the tolerogenic factor is selected from CD46, CD47, CD55, CD59, PD-L1, HLA-E, and HLA-G.

In an embodiment, the cells of the cell population are genetically modified to be lineage restricted. As used herein, the term “lineage restricted” in reference to a cell means that the cell is functionally and/or structurally limited to differentiate into certain cell types.

In an embodiment, the cell population is cryopreserved. In an embodiment, the cell population is cryopreserved in DMSO, or using a cryoprotectant not containing DMSO.

In an embodiment, the cell population comprises at least 1,000 cells, 10,000 cells, 100,000 cells, 1,000,000 cells, or 10,000,000 cells.

### The Cell Population for Use as a Medicament

Another aspect of the present invention relates to an in vitro cell population according to any one of the embodiments described herein or a composition thereof for use as a medicament. In an embodiment, the cell population comprises ventral midbrain neural cells having been contacted with an inhibitor of MEK signaling and an inhibitor of NOTCH signaling. In a preferred embodiment, the cell population is for the treatment of Parkinson's disease.

Another aspect relates to a method of treatment of a neurological condition comprising the administration to a patient of an effective amount of a cell population according to the present invention. In a preferred embodiment, the neurological condition is Parkinson's disease.

Another aspect relates to composition comprising a cell population comprising ventral midbrain NSCs, an inhibitor of MEK signaling, and an inhibitor of NOTCH signaling. In an embodiment, the inhibitor of MEK signaling is Mirdametinib. In an embodiment, the composition is for the treatment of Parkinson's disease. In an embodiment, the cell population comprising ventral midbrain NSCs is characterized by when cultured in vitro for 5 days in a culture medium suitable for maintaining neural cells results in a cell population wherein at least 50% are INA+ or HuCD+ and <15% are K167+. In an embodiment, the cell population is cultured according to Example 6. In an embodiment, the cell population comprising ventral midbrain NSCs, wherein the cell population when cultured in vitro for 5 days in a culture medium suitable for maintaining neural cells results in a cell population wherein at least 25% are HuCD+/SOX2− or HUCD or NEUN+/SOX2−. In an embodiment, the cell population is cultured according to Example 6.

In an embodiment, the composition further comprises a cryoprotectant. In an embodiment, the cryoprotectant is DMSO. In an embodiment, the cryoprotectant does not contain DMSO.

In Vivo Directing Differentiation of Ventral Midbrain Neural Cells into Neurons

Another aspect relates to a method for the treatment of Parkinson's disease, which comprises administering to a subject a therapeutically effective amount of ventral midbrain NSCs and an inhibitor of MEK signaling. In an embodiment, the method further comprises administering an inhibitor of NOTCH signaling. As used herein the term “subject” refers to a human patient suffering from Parkinson's disease. In an embodiment, a “therapeutically effective amount” in reference to a treatment of Parkinson's disease with a cell product means a dose of 200,000 to 10,000,000 cells. Administration to a subject of the cell population comprising ventral midbrain NSCs is contemplated by surgery. In an embodiment, the ventral midbrain NSCs co-express the markers FOXA2, LMX1A, EN1, OTX2, and SOX2. In an embodiment, the ventral midbrain NSCs are obtained according to the methods disclosed herein. Administration of the inhibitor of MEK signaling and/or the inhibitor of NOTCH signaling may be by any suitable means, such as orally or by subcutaneous injection. In an embodiment, administration of the inhibitor of MEK signaling and/or the inhibitor of NOTCH signaling is orally. In a preferred embodiment, the inhibitor of MEK signaling is PD0325901. PD0325901 may also be referred to by the common name Mirdametinib. In an embodiment, the inhibitor of NOTCH signaling is selected from DAPT, MRK-560, MRK-003, LY900009, AL-101, Crenigacestat (LY3039478), MK0752, Nirogacestat (PF-03084017, RO4929097 (RG473), CT16, PTG12, Anti-NRR1, Anti-NRR2, Brontictuzumab (OMP-52M51), Tarextumab (OMP-59R5), 15D11, Anti-Jag1/2, Anti-DII1, YW152F, MMGZO1, mABL001, HMD4-2, Demcizumab (OMP-21M18), Enoticumab (REGN421), MED10639, Navicixizumab (OMP-305B83), ABT-165, NOV1501 (ABL001; HD105), IMR-1, RIN1, SAHM1, and CB-103. In an embodiment, an inhibitor of NOTCH signaling which has been clinically tested is selected. Details of the listed NOTCH inhibitors may be found in Front. Cell Dev. Biol., 28 May 2021, Vol. 9, 2021 (Table 1). In an embodiment, the inhibitor of MEK signaling and/or the inhibitor of NOTCH signaling is administered starting on the same day as the administration of the ventral midbrain NSCs into the patient. In an embodiment, the administration of the inhibitor of MEK signaling and/or the inhibitor of NOTCH signaling is initiated prior to the transplantation of the cells in order to reach steady state in the subject at the time of administration of the cells. In an embodiment, the administration of the inhibitor of MEK signaling and/or the inhibitor of NOTCH signaling is initiated from 6 days prior to transplantation of the cells to 6 days following transplantation of the cells. In an embodiment, the inhibitor of MEK signaling and/or the inhibitor of NOTCH signaling is administered to the subject for at least 2 days, such as at least 3, 4, 5, 6, 7, 8, 9, or 10 days. In an embodiment, the inhibitor of MEK signaling and/or the inhibitor of NOTCH signaling is administered to the subject for 1 to 12 days. The dosage regimen may be any suitable dosage to bring the plasma concentration of the inhibitor of MEK signaling and/or the inhibitor of NOTCH signaling within the effective window for directing differentiation of the administered ventral midbrain NSCs into neurons. In an embodiment, the inhibitor of MEK signaling is Mirdametinib and the dosage regimen is from 1 mg to 30 mg bid (two times a day), 1 mg to 20 mg bid, 1 mg to 10 mg bid, 1 to 5 mg bid, or 2 to 4 mg bid.

In an embodiment, the inhibitor of MEK signaling and/or the inhibitor of NOTCH signaling is co-administered with a cell population comprising ventral midbrain NSCs, such as a cell population having been differentiated for 14 to 24 days, preferably for at least 16 days, optionally, wherein the cell population has not been contacted with an inhibitor of MEK signaling and/or an inhibitor of NOTCH signaling prior to administration into the patient.

It follows that an aspect of the present invention relates to an inhibitor of MEK signaling for use in the treatment of Parkinson's disease in a subject having been administered with a therapeutically effective amount of ventral midbrain NSCs. A further aspect relates to an inhibitor of MEK signaling for use in the treatment of Parkinson's disease in a subject by co-administration with a therapeutically effective amount of ventral midbrain NSCs. In an embodiment, the subject is further co-administrated with an inhibitor of NOTCH signaling. Specifically, an aspect relates to Mirdametinib for use in the treatment of Parkinson's disease. More specifically, an aspect relates to Mirdametinib for use in the treatment of Parkinson's disease in a subject having been administered with a therapeutically effective amount of ventral midbrain NSCs or to Mirdametinib for use in the treatment of Parkinson's disease in a subject by co-administration with a therapeutically effective amount of ventral midbrain NSCs. In a preferred embodiment, the ventral midbrain NSCs co-express the markers FOXA2, LMX1A, EN1, OTX2, and SOX2.

Another aspect relates to an inhibitor of MEK signaling and an inhibitor of NOTCH signaling for use in combination in the treatment of Parkinson's disease in a subject having been administered with a therapeutically effective amount of ventral midbrain NSCs. Similarly, an aspect relates to an inhibitor of MEK signaling and an inhibitor of NOTCH signaling for use in combination in a method for directing differentiation of ventral midbrain NSCs into ventral midbrain neurons, wherein the method comprises administering the inhibitor of MEK signaling and the inhibitor of NOTCH signaling in combination to a subject having been administered with the ventral midbrain NSCs. In an embodiment, the inhibitor of MEK signaling and the inhibitor of NOTCH signaling is administered at least within 6 days following the administration of the ventral midbrain NSCs. In an embodiment, the inhibitor of MEK signaling and the inhibitor of NOTCH signaling is administered for at least 2 days, such as for at least 4, 5, or 6 days.

A further aspect relates to an inhibitor of MEK signaling, an inhibitor of NOTCH signaling, and a cell population comprising ventral midbrain NSCs for use in combination in the treatment of Parkinson's disease. In an embodiment, the inhibitor of MEK signaling and the inhibitor of NOTCH signaling is co-administered together with the cell population comprising ventral midbrain NSCs. In an embodiment, the inhibitor of MEK signaling is Mirdametinib.

Another aspect of the present invention relates to a composition comprising a cell population comprising ventral midbrain NSCs and an inhibitor of MEK signaling. In an embodiment, the inhibitor of MEK signaling is Mirdametinib. In an embodiment, the composition further comprises an inhibitor of NOTCH signaling. In a specific embodiment, the inhibitor of NOTCH signaling is DAPT. Another aspect relates to a composition comprising a cell population comprising ventral midbrain NSCs, an inhibitor of MEK signaling, and an inhibitor of NOTCH signaling. In an embodiment, the composition is in vitro.

A person skilled in the art will appreciate that the embodiments referred to in this section relating to methods of treatment, use as a medicament and second medical uses may equally apply to all aspects mentioned.

### Cell Population for Use in the Treatment of Parkinson's Disease

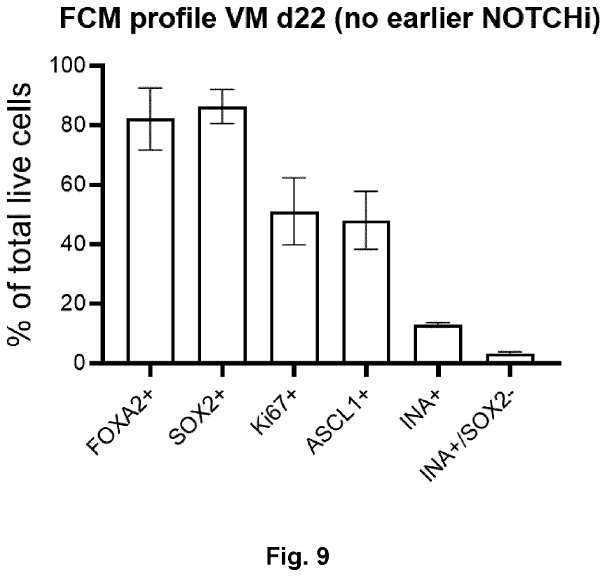

The present inventors realized that obtaining a cell population comprising ventral midbrain neural cells without contacting the cell population with an inhibitor of NOTCH, such as DAPT, reflects better the natural development of the neural cells. While the present inventors would still prefer further contacting the cell population with the inhibitor of MEK signaling and the inhibitor of NOTCH signaling, the cell population having the preferred expression profile at the time of contacting the cells with these inhibitors may in themselves be more suitable for treatment of Parkinson's Disease as compared to prior art methods. Accordingly, another aspect of the present invention relates to a cell population for use in the treatment of Parkinson's Disease, wherein 80-95% of the cell population express SOX2, 40-60% of the cell population express ASCL1, 30-65% of the cell population express KI67, 10-20% of the cell population express INA, and 2-5% of the cell population are INA+/SOX2-(FIG. 9, Table 3). In a preferred embodiment, the cell population has not been contacted with DAPT or other inhibitors of NOTCH signaling prior to contact with both MEK inhibitors and NOTCH inhibitors. These cells are obtained according to the methods disclosed herein, prior to contacting the cell population with the inhibitors, wherein the cell population is allowed to differentiate into ventral midbrain NSCs for at least 20 days, such 22-24 days.

The cell population may be administered to a patient on its own or co-delivered together with an inhibitor of MEK signaling and/or an inhibitor of NOTCH signaling. Accordingly, an aspect relates to composition comprising the aforementioned cell population and an inhibitor of MEK signaling and/or an inhibitor of NOTCH signaling.

### PARTICULAR EMBODIMENTS

The aspects of the present invention are now further described by the following non-limiting embodiments:
1. A method comprising contacting a cell population comprising ventral midbrain NSCs with an inhibitor of MEK signaling and an inhibitor of NOTCH signaling.
2. The method according to the preceding embodiment, wherein the method is for directing differentiation of ventral midbrain NSCs into neurons.
3. The method according to any one of the preceding embodiments, wherein the method is for differentiating ventral midbrain NSCs into ventral midbrain neurons.
4. The method according to any one of the preceding embodiments, wherein the ventral midbrain NSCs are neurally induced, ventralized, and caudalized.
5. The method according to any one of the preceding embodiments, wherein the cell population is neurally induced, ventralized, and caudalized to obtain ventral midbrain NSCs.
6. The method according to any one of the preceding embodiments, wherein the inhibitor of MEK signaling inhibits MEK1 and MEK2.
7. The method according to any one of the preceding embodiments, wherein the inhibitor of NOTCH signaling targets y-secretase.
8. The method according to any one of the preceding embodiments, wherein at least 5% of the cell population comprising ventral midbrain NSCs co-express the markers FOXA2, LMX1A, EN1, OTX2, and SOX2.
9. The method according to any one of the preceding embodiments, wherein the cell population is allowed to differentiate into ventral midbrain NSCs for 14 to 24 days prior to contacting the cell population with the inhibitor of MEK signaling and the inhibitor of NOTCH signaling.
10. The method according to any one of the embodiments 5 to 9, wherein the cell population comprising ventral midbrain NSCs is contacted with the inhibitor of MEK signaling and the inhibitor of NOTCH signaling prior to more than 70%, or 60%, of the cell population having further developed into intermediate neural precursors.
11. The method according to any one of the preceding embodiments, wherein 0.5-65%, preferably 30-65%, more preferably 40-60%, of the cell population comprising ventral midbrain NSCs express ASCL1 at the time of contacting the cell population with the inhibitor of MEK signaling and the inhibitor of NOTCH signaling.
12. The method according to any one of the preceding embodiments, wherein 25-65%, preferably 45-65%, of the cell population comprising ventral midbrain NSCs express KI67 at the time of contacting the cell population with the inhibitor of MEK signaling and the inhibitor of NOTCH signaling.
13. The method according to any one of the preceding embodiments, wherein less than 20%, preferably less than 15%, of the cell population comprising ventral midbrain NSCs express INA at the time of contacting the cell population with the inhibitor of MEK signaling and the inhibitor of NOTCH signaling.
14. The method according to any one of the preceding embodiments, wherein 0-20%, preferably 10-15%, of the cell population comprising ventral midbrain NSCs express INA at the time of contacting the cell population with the inhibitor of MEK signaling and the inhibitor of NOTCH signaling.
15. The method according to any one of the preceding embodiments, wherein less than 10%, preferably less than 5% of the cell population comprising ventral midbrain NSCs are INA+/SOX2− at the time of contacting the cell population with the inhibitor of MEK signaling and the inhibitor of NOTCH signaling.
16. The method according to any one of the preceding embodiments, wherein 0-10%, preferably 2-5%, of the cell population comprising ventral midbrain NSCs are INA+/SOX2− at the time of contacting the cell population with the inhibitor of MEK signaling and the inhibitor of NOTCH signaling.
17. The method according to any one of the preceding embodiments, wherein 75-95%, preferably 80-95% of the cell population comprising ventral midbrain NSCs express SOX2 at the time of contacting the cell population with the inhibitor of MEK signaling and the inhibitor of NOTCH signaling.
18. The method according to any one of the preceding embodiments 5 to 10, wherein the cell population comprising ventral midbrain NSCs is contacted with the inhibitor of MEK signaling and the inhibitor of NOTCH signaling prior to more than 20% of the cell population having further developed into intermediate neural precursors or neurons.
19. The method according to any one of the embodiments 5 to 10 and 18, wherein the cell population comprising ventral midbrain NSCs is contacted with the inhibitor of MEK signaling and the inhibitor of NOTCH signaling prior to more than 20% of the cell population expresses one of the markers ASCL1 and INA.
20. The method according to any one of the preceding embodiments, wherein the inhibitor of MEK signaling is selected from PD0325901, trametinib (GSK1120212), selumetinib (AZD6244), pimasertib (AS703026), MEK162, cobimetinib, PD184352, PD173074, BIX02189, AZD8330, PD318088, Refametinib, and PD98059, preferably PD0325901.
21. The method according to any one of the preceding embodiments, wherein the concentration of the inhibitor of MEK signaling is at least 5 μM, preferably at least 10 μM.
22. The method according to any one of the preceding embodiments, wherein the concentration of the inhibitor of MEK signaling is from 5 μM to 100 μM.
23. The method according to any one of the preceding embodiments, wherein the inhibitor of NOTCH signaling is selected from DAPT, Avagacestat, PF-03084014, and LY450139.
24. The method according to any one of the preceding embodiments, wherein the inhibitor of NOTCH signaling is selected from DAPT, MRK-560, MRK-003, LY900009, AL-101, Crenigacestat (LY3039478), MK0752, Nirogacestat (PF-03084017, R04929097 (RG473), CT16, PTG12, Anti-NRR1, Anti-NRR2, Brontictuzumab (OMP-52M51), Tarextumab (OMP-59R5), 15D11, Anti-Jag1/2, Anti-DII1, YW152F, MMGZ01, mABL001, HMD4-2, Demcizumab (OMP-21M18), Enoticumab (REGN421), MED10639, Navicixizumab (OMP-305B83), ABT-165, NOV1501 (ABL001; HD105), IMR-1, RIN1, SAHM1, and CB-103.
25. The method according to any one of the preceding embodiments, wherein the concentration of the inhibitor of NOTCH signaling is at least 1 μM, preferably at least 10 μM.
26. The method according to any one of the preceding embodiments, wherein the concentration of the inhibitor of NOTCH signaling is from 1 μM to 100 μM.
27. The method according to any one of the preceding embodiments, wherein the cell population is contacted with the inhibitor of MEK signaling for at least 2 hour, 1 hour, 2, 3, 4, 5, 6, 7, 8, 9, 10, 11, or 12 hours, or 18 hours, or 24 hours, or for at least 2 days, 3 days, 4 days, 5 days, or 6 days.
28. The method according to any one of the preceding embodiments, wherein the cell population is contacted with the inhibitor of MEK signaling for 2 hour to 15 days, % hour to 10 days, % hour to 5 days, 2 hour to 2 days, 1 hour to 2 days, 3 hours to 2 days, 6 hours to 2 days, 12 hours to 2 days, 18 hours to 2 days, or 1 to 2 day(s), or for about 2 to about 15 days.
29. The method according to any one of the preceding embodiments, wherein the cell population is contacted with the inhibitor of NOTCH signaling for at least % hour, 1 hour, 2, 3, 4, 5, 6, 7, 8, 9, 10, 11, or 12 hours, or 18 hours, or 24 hours, or for at least 2 days, 3 days, 4 days, 5 days, or 6 days.
30. The method according to any one of the preceding embodiments, wherein the cell population is contacted with the inhibitor of NOTCH signaling for %2 hour to 15 days, % hour to 10 days, % hour to 5 days, % hour to 2 days, 1 hour to 2 days, 3 hours to 2 days, 6 hours to 2 days, 12 hours to 2 days, 18 hours to 2 days, or 1 to 2 day(s), or for about 2 to about 15 days.
31. The method according to any one of the preceding embodiments, wherein the inhibitor of MEK signaling and the inhibitor of NOTCH signaling are replaced at least every 36 hours, preferably at least every 30 hours, more preferably at least every 24 hours.
32. The method according to any one of the preceding embodiments, wherein the inhibitor of MEK signaling and the inhibitor of NOTCH signaling are replaced at least every 12 to 36 hours, preferably at least every 18 to 30 hours, more preferably at least every 24 hours.
33. The method according to any one of the preceding embodiments, wherein the cell population is contacted with the inhibitor of MEK signaling and the inhibitor of NOTCH signaling at least partly at the same time.
34. The method according to any one of the preceding embodiments, wherein the cell population is contacted with the inhibitor of MEK signaling and the inhibitor of NOTCH signaling simultaneously.
35. The method according to any one of the preceding embodiments, wherein the cell population is not contacted with an inhibitor of NOTCH signaling prior to the contacting of the cell population with the inhibitor of MEK signaling and the inhibitor of NOTCH signaling according to the method.
36. The method according to any one of the preceding embodiments, wherein the cell population is cryopreserved following the inhibition of MEK and NOTCH signaling.
37. The method according to the preceding embodiment, wherein the cell population is cryopreserved within 4 days, such as within 3 days, 2 days, 1 day, 12 hours, 6 hours, or 3 hours, following end of inhibition of MEK and NOTCH signaling.
38. The method according to the preceding embodiment, wherein the cell population is cryopreserved immediately following end of inhibition of MEK and NOTCH signaling.
39. The method according to any one of the embodiments 36 to 38, wherein the cell population is cryopreserved in DMSO, or using a cryoprotectant not containing DMSO.
40. The method according to any one of the preceding embodiments, wherein prior to contacting the cell population with the inhibitor of MEK signaling and the inhibitor of NOTCH signaling, the cell population is cultured to induce differentiation into ventral midbrain NSCs.
41. The method according to any one of the preceding embodiments, wherein prior to contacting the cell population with the inhibitor of MEK signaling and the inhibitor of NOTCH signaling, the cell population is contacted with an inhibitor of Small Mothers Against Decapentaplegic (SMAD) protein signaling, an activator of Sonic Hedgehog (SHH) signaling, an activator of wingless (Wnt) signaling, and/or an activator of fibroblast growth factor (FGF) signaling, and, optionally, ascorbic acid and/or, optionally, Brain-derived neurotrophic factor (BDNF).
42. The method according to the preceding embodiment, wherein the cell population is contacted with at least two inhibitors of SMAD protein signaling.
43. The method according to any one of the embodiments 41 and 42, wherein the inhibitor(s) of SMAD signaling is selected from Noggin, LY364947, SB431542, RepSox, and LDN-193189, or a similar compound.
44. The method according to any one of the embodiments 41 to 43, wherein the activator of SHH signaling is selected from SHH, purmorphamine, SAG, or a similar compound.
45. The method according to any one of the embodiments 41 to 44, wherein the activator of Wnt signaling is CHIR99021.
46. The method according to any one of the embodiments 41 to 45, wherein the activator of FGF signaling is FGF8b.
47. The method according to any one of the embodiments 41 to 46, wherein the concentration of:
   a) the inhibitor(s) of SMAD protein signaling is from 1 μM to 50 μM,
   b) the activator of SHH signaling is from 200 ng/ml to 800 ng/ml,
   c) the inhibitor of Wnt signaling is from 0.1 μM to 1 μM,
   d) the activator of FGF signaling is from 10 ng/ml to 200 ng/ml,
   e) ascorbic acid is from 50 μM to 500 μM, and/or
   f) BDNF is from 1 ng/ml to 50 ng/ml.
48. The method according to any one of the preceding embodiments, wherein the cell population is derived from PSCs.
49. The method according to the preceding embodiment, wherein the PSCs are human embryonic stem cells or human induced pluripotent stem cells.
50. The method according to any one of the preceding embodiments, wherein the method is in vitro.
51. An in vitro method for directing differentiation of a cell population of PSCs into ventral midbrain neurons, comprising culturing the cell population of PSCs, contacting the cell population of PSCs with an inhibitor of SMAD protein signaling, an inhibitor of Wnt signaling, an activator of SHH signaling, an activator of FGF signaling, optionally ascorbic acid, and, optionally BDNF, to obtain a cell population comprising ventral midbrain NSCs, wherein the cell population comprising ventral midbrain NSCs is further contacted with an inhibitor of MEK signaling and an inhibitor of NOTCH signaling to direct differentiation into ventral midbrain neurons.
52. The method according to the preceding embodiment, wherein the cell population is allowed to differentiate into ventral midbrain NSCs for 14 to 24 days prior to contacting the cell population with the inhibitor of MEK signaling and the inhibitor of NOTCH signaling.
53. The method according to any one of the embodiments 51 and 52, wherein the cell population is harvested before 28 days from beginning differentiation of the cell population into ventral midbrain NSCs, such as before 27 days, 26 days, or 25 days, preferably before 26 days.
54. The method according to any one of the embodiments 51 to 53, wherein the cell population is harvested at day 28, day 27, day 26, or day 25 from beginning differentiation of the cell population into ventral midbrain NSCs, preferably at day 25.
55. The method according to any one of the embodiments 51 to 54, wherein the cell population is contacted with the cell population with the inhibitor of MEK signaling and the inhibitor of NOTCH signaling for %2 hour to 15 days, % hour to 10 days, % hour to 5 days, %2 hour to 2 days, 1 hour to 2 days, 3 hours to 2 days, 6 hours to 2 days, 12 hours to 2 days, 18 hours to 2 days, or 1 to 2 day(s), or for about 2 to about 10 days.
56. The method according to any one of the embodiments 51 to 55, wherein the cell population is contacted with the inhibitor of MEK signaling and the inhibitor of NOTCH signaling from 0 to 10 days after the cell population is no longer contacted with the activator of FGF signaling.
57. The method according to any one of the embodiments 51 to 56, wherein prior to contacting the cell population with the inhibitor of MEK signaling and the inhibitor of NOTCH signaling, the cell population is neurally induced, ventralized and caudalized for 16 days, followed by culturing for 6 to 8 days in a suitable culture medium without contacting the cell population with an inhibitor of SMAD protein signaling, an inhibitor of Wnt signaling, an activator of SHH signaling, an activator of FGF signaling, or an inhibitor of NOTCH signaling, such as DAPT.
58. The method according to any one of the preceding embodiments 51 to 57, wherein the cell population is contacted with the inhibitor of MEK signaling and the inhibitor of NOTCH signaling at least partly at the same time.
59. The method according to any one of the embodiments 51 to 58, wherein the cell population is contacted with the inhibitor of MEK signaling and the inhibitor of NOTCH signaling simultaneously.
60. The method according to any one of the embodiments 51 to 59, wherein the cell population of pluripotent stem cells is contacted with the inhibitor of SMAD protein signaling for 5 to 9 days.
61. The method according to any one of the embodiments 51 to 60, wherein the cell population of pluripotent stem cells is contacted with the inhibitor of Wnt signaling for 5 to 9 days.
62. The method according to any one of the embodiments 51 to 61, wherein the cell population of pluripotent stem cells is contacted with the activator of SHH signaling for 5 to 9 days.
63. The method according to any one of the embodiments 51 to 62, wherein the cell population of pluripotent stem cells is contacted with the activator of FGF signaling for 7 to 12 days following ended contacting with the inhibitor of SMAD protein signaling, inhibitor of Wnt signaling, and/or activator of SHH signaling.
64. The method according to any one of the embodiments 51 to 63, wherein the cell population is contacted with the inhibitor of SMAD protein signaling for 5 to 9 days from day 0.
65. The method according to any one of the embodiments 51 to 64, wherein the cell population is contacted with the inhibitor of Wnt signaling for 5 to 9 days from day 0.
66. The method according to any one of the embodiments 51 to 65, wherein the cell population is contacted with the activator of SHH signaling for 5 to 9 days from day 0.
67. The method according to any one of the embodiments 51 to 66, wherein the cell population is contacted with the activator of FGF signaling for 7 to 12 days from day 5 to 9, or when ending contacting with the inhibitor of SMAD protein signaling, inhibitor of Wnt signaling, and/or activator of SHH signaling.
68. The method according to any one of the embodiments 51 to 67, wherein the cell population is contacted with ascorbic acid for 5 to 7 days from day 10 or 11.
69. The method according to any one of the embodiments 51 to 68, wherein the concentration of ascorbic acid is from 10 μM to 400 μM.
70. The method according to any one of the embodiments 51 to 69, wherein the cell population is contacted with BDNF for 5 to 7 days from day 10 or 11.
71. The method according to any one of the embodiments 51 to 70, wherein the concentration of BDNF is from 1 ng/ml to 40 ng/ml.
72. The method according to any one of the embodiments 51 to 71, wherein the cell population is cryopreserved following the inhibition of MEK signaling and inhibition of NOTCH signaling.
73. The method according to the preceding embodiment, wherein the cell population is cryopreserved within 4 days, such as within 3 days, 2 days, 1 day, 12 hours, 6 hours, 3 hours, or 1 hour, following end of inhibition of MEK signaling and inhibition of NOTCH signaling.
74. The method according to any one of the embodiments 72 and 73, wherein the cell population is cryopreserved at harvest.
75. The method according to the preceding embodiment, wherein the cell population is cryopreserved within 4 days, such as within 3 days, 2 days, 1 day, 12 hours, 6 hours, or 3 hours, following harvest.
76. The method according to any one of the embodiments 51 to 75, wherein 0.5-65%, preferably 30-65%, more preferably 40-60%, of the cell population comprising ventral midbrain NSCs express ASCL1 at the time of contacting the cell population with the inhibitor of MEK signaling and the inhibitor of NOTCH signaling.
77. The method according to any one of the embodiments 51 to 76, wherein 25-65%, preferably 45-65%, of the cell population comprising ventral midbrain NSCs express KI67 at the time of contacting the cell population with the inhibitor of MEK signaling and the inhibitor of NOTCH signaling.
78. The method according to any one of the embodiments 51 to 77, wherein less than 20%, preferably less than 15%, of the cell population comprising ventral midbrain NSCs express INA at the time of contacting the cell population with the inhibitor of MEK signaling and the inhibitor of NOTCH signaling.
79. The method according to any one of the embodiments 51 to 78, wherein 0-20%, preferably 10-15%, of the cell population comprising ventral midbrain NSCs express INA at the time of contacting the cell population with the inhibitor of MEK signaling and the inhibitor of NOTCH signaling.
80. The method according to any one of the embodiments 51 to 79, wherein less than 10%, preferably less than 5% of the cell population comprising ventral midbrain NSCs are INA+/SOX2− at the time of contacting the cell population with the inhibitor of MEK signaling and the inhibitor of NOTCH signaling.
81. The method according to any one of the embodiments 51 to 80, wherein 0-10%, preferably 2-5%, of the cell population comprising ventral midbrain NSCs are INA+/SOX2− at the time of contacting the cell population with the inhibitor of MEK signaling and the inhibitor of NOTCH signaling.
82. The method according to any one of the embodiments 51 to 81, wherein 75-95%, preferably 80-95% of the cell population comprising ventral midbrain NSCs express SOX2 at the time of contacting the cell population with the inhibitor of MEK signaling and the inhibitor of NOTCH signaling.
83. An in vitro method for directing differentiation of a cell population into ventral midbrain neurons, comprising the steps of:
   culturing a cell population comprising PSCs,
   inducing differentiation of the cell population into ventral midbrain NSCs,
   allowing the ventral midbrain NSCs to mature, and
   contacting the cell population comprising ventral midbrain NSCs with an inhibitor of NOTCH signaling,
   wherein the ventral midbrain NSCs express the markers FOXA2, LMX1A, EN1, OTX2, and SOX2.
84. The method according to the preceding embodiment, wherein the cell population is contacted with the inhibitor of NOTCH signaling at least 20 days, preferably 21 days, more preferably 22 days, after initially inducing differentiation of the cell population into ventral midbrain NSCs.
85. A cell population comprising ventral midbrain neural cells obtainable by the method according to any one of the embodiments 1 to 84.
86. A cell population comprising ventral midbrain neural cells obtained by the method according to any one of the embodiments 1 to 84.
87. A cell population comprising ventral midbrain NSCs, wherein the cell population when cultured in vitro for 5 days in a culture medium suitable for maintaining neural cells results in a cell population wherein at least 50% are INA+ or HUCD+, and less than 15% are KI67+.
88. A cell population comprising ventral midbrain NSCs, wherein the cell population when cultured in vitro for 5 days in a culture medium suitable for maintaining neural cells results in a cell population wherein at least 25% are HuCD+/SOX2− or HUCD+ or NEUN+/SOX2−.
89. The cell population according to any one of the embodiments 87 and 88, wherein the expression of markers by the cells is measured using single cell RNA sequencing, nucleic RNA sequencing, immunocytochemistry (ICC), qPCR, or FACS.
90. The cell population according to any one of the embodiments 87 to 89, wherein the expression of markers by the cells is measured according to Example 7.
91. The cell population according to any one of the embodiments 87 to 89, wherein the expression of markers by the cells is measured according to Example 8
92. The cell population according to any one of the embodiments 87 to 89, wherein the expression of markers by the cells is measured according to Example 9.
93. The cell population according to any one of the embodiments 87 to 92, wherein the cell population is cultured according to Example 6.
94. The cell population according to any one of the embodiments 85 to 93, wherein the cell population is in vitro.
95. The cell population according to any one of the embodiments 85 to 94, wherein the ventral midbrain neural cells are non-native.
96. The cell population according to any one of the embodiments 85 to 95, wherein the ventral midbrain neural cells are artificial.
97. The cell population according to any one of the embodiments 85 to 96, wherein the ventral midbrain neural cells are stem cell-derived.
98. The cell population according to any one of the embodiments 85 to 97, wherein the ventral midbrain neural cells are stem cell-derived from pluripotent stem cells.
99. The cell population according to the preceding embodiment, wherein the pluripotent stem cells are genetically modified and wherein the genetic modifications persist in the ventral midbrain neural cells.
100. The cell population according to any one of the embodiments 98 and 99, wherein the ventral midbrain neural cells are stem cell-derived from human embryonic stem cells (hESCs) or human induced pluripotent stem cells (hiPSCs).
101. The cell population according to any one of the embodiments 85 to 100, wherein the cells of the cell population are genetically modified.
102. The cell population according to the preceding embodiment, wherein the cells of the cell population are genetically modified to be hypoimmunogenic and/or lineage restricted.
103. The cell population according to the preceding embodiment, wherein the genetic modifications for hypoimmunogenicity comprise reduced expression of MHC-I human leukocyte antigens relative to a wild-type stem cell, reduced expression of MHC-II human leukocyte antigens relative to a wild-type stem cell, and/or increased expression of a tolerogenic factor relative to wild-type stem cell.
104. The cell population according to the preceding embodiment, wherein the MHC-I human leukocyte antigens are HLA-A, HLA-B, and HLA-C.
105. The cell population according to any one of the embodiments 103 and 104, wherein the MHC-II human leukocyte antigens are HLA-DP, HLA-DQ, and HLA-DR.
106. The cell population according to any one of the embodiments 103 to 105, wherein the tolerogenic factor is selected from CD46, CD47, CD55, CD59, PD-L1, HLA-E, and HLA-G.
107. The cell population according to any one of the embodiments 85 to 106, wherein the cell population comprises at least 1,000 cells, 10,000 cells, 100,000 cells, 1,000,000 cells, or 10,000,000 cells.
108. The cell population according to any one of the embodiments 85 to 107, wherein the cell population is cryopreserved.
109. A composition comprising the cell population according to any one of the embodiments 85 to 108.
110. The composition according to the preceding embodiment, further comprising a cryoprotectant.
111. The composition according to the preceding embodiment, wherein the cryoprotectant is DMSO, or wherein the cryoprotectant does not contain DMSO.
112. An in vitro cell population according to any one of the embodiments 85 to 108 for use as a medicament.
113. The in vitro cell population according to the preceding embodiment, for the treatment of Parkinson's disease.
114. A method of treatment of a neurological condition comprising the administration to a patient of an effective amount of a cell population according to any one of the embodiments 85 to 108.
115. The method according to the preceding embodiment, wherein the neurological condition is Parkinson's disease.
116. A method for treatment of Parkinson's disease, which comprises administering to a subject a therapeutically effective amount of ventral midbrain NSCs and an inhibitor of MEK signaling.
117. The method according to the preceding embodiment, wherein the inhibitor of MEK signaling is PD0325901.
118. The method according to any one of the embodiments 116 and 117, wherein the ventral midbrain NSCs co-express the markers FOXA2, LMX1A, EN1, OTX2, and SOX2.
119. The method according to any one of the embodiments 116 to 118, which further comprises administering an inhibitor of NOTCH signaling.
120. The method according to the preceding embodiment, wherein the inhibitor of NOTCH signaling is selected from DAPT, MRK-560, MRK-003, LY900009, AL-101, Crenigacestat (LY3039478), MK0752, Nirogacestat (PF-03084017, R04929097 (RG473), CT16, PTG12, Anti-NRR1, Anti-NRR2, Brontictuzumab (OMP-52M51), Tarextumab (OMP-59R5), 15D11, Anti-Jag1/2, Anti-DII1, YW152F, MMGZ01, mABL001, HMD4-2, Demcizumab (OMP-21M18), Enoticumab (REGN421), MED10639, Navicixizumab (OMP-305B83), ABT-165, NOV1501 (ABL001; HD105), IMR-1, RIN1, SAHM1, and CB-103.
121. The method according to the preceding embodiment, wherein the inhibitor of NOTCH signaling is DAPT.
122. An inhibitor of MEK signaling and an inhibitor of NOTCH signaling for use in combination in the treatment of Parkinson's disease in a subject having been administered with a therapeutically effective amount of ventral midbrain NSCs.
123. The inhibitor of MEK signaling and the inhibitor of NOTCH signaling according to the preceding embodiment, wherein the inhibitor of MEK signaling and the inhibitor of NOTCH signaling is administered at least within 6 days following the administration of the ventral midbrain NSCs.
124. The inhibitor of MEK signaling and the inhibitor of NOTCH signaling in combination according any one of the embodiments 122 and 123, wherein the inhibitor of MEK signaling and the inhibitor of NOTCH signaling is administered for at least 2 days, such as for at least 4, 5, or 6 days.
125. An inhibitor of MEK signaling, an inhibitor of NOTCH signaling, and a cell population comprising ventral midbrain NSCs for use in combination in the treatment of Parkinson's disease.
126. The inhibitor of MEK signaling, the inhibitor of NOTCH signaling and the cell population according to the preceding embodiment, wherein the inhibitor of MEK signaling and the inhibitor of NOTCH signaling is co-administered together with the cell population comprising ventral midbrain NSCs.
127. The inhibitor of MEK signaling, the inhibitor of NOTCH signaling and the cell population according to the preceding embodiment, wherein the inhibitor of MEK signaling is Mirdametinib.
128. An inhibitor of MEK signaling for use in the treatment of Parkinson's disease in a subject having been administered with a therapeutically effective amount of ventral midbrain NSCs.
129. The inhibitor of MEK signaling according to the preceding embodiment, wherein the ventral midbrain NSCs have been administered by surgery.
130. Mirdametinib for use in the treatment of Parkinson's disease.
131. Mirdametinib for use in the treatment of Parkinson's disease in a subject having been administered with a therapeutically effective amount of ventral midbrain NSCs.
132. A composition comprising a cell population comprising ventral midbrain NSCs, an inhibitor of MEK signaling, and an inhibitor of NOTCH signaling.
133. The composition according to the preceding embodiment, wherein the inhibitor of MEK signaling is Mirdametinib.
134. The composition according to any one of the embodiments 132 and 133, for the treatment of Parkinson's disease.
135. The composition according to any one of the embodiments 132 to 134, wherein the cell population comprising ventral midbrain NSCs is according to embodiment 87 and/or 88.
136. The composition according to any one of the embodiments 132 to 135, wherein the composition is in vitro.
137. An in vitro cell population for use in the treatment of Parkinson's Disease, wherein 80-95% of the cell population express SOX2, 40-60% of the cell population express ASCL1, 30-65% of the cell population express K167, 10-20% of the cell population express INA, and 2-5% of the cell population are INA+/SOX2−.
138. The cell population according to the preceding embodiment, wherein the cell population has not been contacted with DAPT.
139. A composition comprising a cell population according to embodiment 137 or 138, and an inhibitor of MEK signaling and/or an inhibitor of NOTCH signaling.

### EXAMPLES

The following are non-limiting examples for carrying out the invention.

### Example 1: Differentiation of Human Pluripotent Stem Cells to Ventral Midbrain Neural Cells

hPSCs can be differentiated to ventral midbrain neural cells according to several protocols that have been published since 2011 when the seminal paper from Lorenz Studer's research group was published, titled “Dopamine neurons derived from human ES cells efficiently engraft in animal models of Parkinson's disease”. One way to perform the differentiation procedure is shown below, following the publications described by the references in this section (Nolbrant et al., 2017 and Kirkeby et al., 2017).

Human embryonic stem cell (hESCs) lines RC17 (Roslin CT) and 3053 (Novo Nordisk A/S) and other hESC lines developed by Novo Nordisk, were cultured in iPS Brew XF media (Miltenyi Biotec) supplemented with 60 U/mL Penicillin-Streptomycin (P-S; Thermo Fisher Scientific) on human laminin-521 matrix (0.7-1.2 μg/cm²; Biolamina) coated culture ware. Media was changed daily, and cells passaged with EDTA 0.5 mM (Thermo Fisher Scientific) every 4-6 days. Cultures were maintained at 37° C., humidity 95% and a 5% CO₂ level.

hESCs were differentiated to ventral midbrain neurons according to an established protocol (Nolbrant et al., 2017; Kirkeby et al., 2017). In brief, hESC were grown to 70-90% confluency, then disassociated with 0.5 mM EDTA. The cells were seeded at 10⁴ cells/cm² in cell culture flasks or plates coated with human laminin-111 (1.2 μg/cm²; BioLamina) and immediately put into contact with differentiation media. The cells were exposed to N2-based media from days in vitro (DIV) 0-8; 50% DMEM/F12+Glutamax (Gibco), 50% Neurobasal (Gibco), 1% N2 supplement CTS (Thermo Fisher Scientific), 5% GlutaMAX (Thermo Fisher Scientific), 0.2% P-S (Thermo Fisher Scientific) and supplemented with SMAD inhibitors SB431542 (10 μM; Miltenyi Biotec), Noggin (100 ng/mL; Miltenyi Biotec) for neural induction, Sonic Hedgehog C24II (SHH; 500 ng/mL; Miltenyi Biotec) for ventral fate, GSK3B inhibitor CHIR99021 (CHIR; 0.5-0.6 μM; Miltenyi Biotec) to promote caudalization. N2-based media was supplemented with fibroblast growth factor 8b (FGF8b; 100 ng/mL; Miltenyi Biotec) from DIV9-11. At DIV11, the cells were dissociated with accutase (Thermo Fisher Scientific) and seeded at 0.8×10⁶ cells/m² in a cell culture flask or plate coated with human laminin-111 (1.2 μg/cm²) in DIV11-16 media (Neurobasal, 2% B27 supplement without vitamin A CTS (Thermo Fisher Scientific), 5% GlutaMAX, 0.2% P-S and supplemented with FGF8b (100 ng/mL), L-ascorbic acid (AA; 200 μM; Sigma), human Brain Derived Neurotrophic Factor (BDNF; 20 ng/mL; Miltenyi Biotec)) supplemented with Y-27632 (Miltenyi Biotec) at 10 μM. At day in vitro (DIV) 16, the cells were dissociated with accutase and either cryopreserved, or re-seeded in cell culture flasks/plates coated with poly-L-ornithine (0.002%) and Laminin-521 (1.5 μg/cm²) in B27 media supplemented with BDNF (20 ng/mL), GDNF (20 ng/mL), L-ascorbic acid (200 μM), dcAMP (500 μM), DAPT (10 μM), and Y-27632 (10 μM) for extended in vitro culture allowing further differentiation and maturation of ventral midbrain neural stem cells into neurons.

### REFERENCES

Nolbrant S, Heuer A, Parmar M, Kirkeby A. Generation of high-purity human ventral midbrain dopaminergic progenitors for in vitro maturation and intracerebral transplantation. Nat Protoc. 2017 September; 12(9):1962-1979. doi: 10.1038/nprot.2017.078. Epub 2017 Aug. 31. PMID: 28858290.
Kirkeby A, Nolbrant S, Tiklova K, Heuer A, Kee N, Cardoso T, Ottosson D R, Lelos M J, Rifes P, Dunnett S B, Grealish S, Perlmann T, Parmar M. Predictive Markers Guide Differentiation to Improve Graft Outcome in Clinical Translation of hESC-Based Therapy for Parkinson's Disease. Cell Stem Cell. 2017 Jan. 5; 20(1):135-148. doi: 10.1016/j.stem.2016.09.004. Epub 2016 Oct. 27. PMID: 28094017; PMCID: PMC5222722.

### Example 2: Flow Cytometry Analytical Single Cell Protein Marker Expression Assay

Ventral midbrain dopaminergic (vmDA) progenitor cells were generated from hESCs in 2D in vitro culture as described in Example 1 and using reagents described in Table 1. At various days after initiating the differentiation the cell culture was dissociated into a single cell suspension using Accutase, counted on a NucleoCounter NC-200 and collected in N2 media (CTS™ Neurobasal™ medium supplemented with 1% CTS™ N-2 supplement). Dead cells were labelled using a LIVE/DEAD™ Fixable Near-IR Dead Cell Stain Kit. The cells were then resuspended in B27 media (CTS™ Neurobasal™ medium supplemented with 1% B-27™ supplement without vitamin A, 2 mM GSutaMAX™, 60 U/mL Penicillin-Streptomycin, 10 μM ROCK inhibitor). Cells were then fixed and permeabilized using the BD Transcription Factor Buffer Set (BD Biosciences) according to the manufacturer's instructions. The fixed cells were then stained with fluorescently conjugated antibodies, and the samples acquired on a BD LSR Fortessa or B FACSymphony (BD Biosciences). The fcs files were exported and analyzed in FlowJo 10.5.03.

To determine whether a cell was considered positive in its protein expression of a marker, gates were set as routinely done in the field at the edge of the fluorescent signal of a negative control sample. Examples of negative control samples used included unstained controls, fluorescence minus one (FMO) controls and most preferably biological negative control samples; all cells present above these threshold gates when experimental samples were run were considered positive. Antibodies: FOXA2 (1:320, Miltenyi), OTX2 (1:320, Miltenyi), SOX2 (1:40, B), LMXTA (1:2500, Novo Nordisk production), EN9 (1:40, AtlasAntibodies), ASCL1 (1:2500, Mitlenyi), INA (1:3000, AtlasAntibodies).

TABLE 1
List of reagents used for FACS, ICC and cell culture

| | | Catalog |
|---|---|---|
| Reagent | Supplier | number |
| CTS ™ Neurobasal ™ medium | Thermo Fisher Scientific | A1371201 |
| CTS ™ N-2 supplement | Thermo Fisher Scientific | A1370701 |
| B-27 ™ Supplement, XenoFree, minus | Thermo Fisher Scientific | A3353501 |
| vitamin A | | |
| GlutaMAX ™ Supplement | Thermo Fisher Scientific | 35050061 |
| Penicillin-Streptomycin | Thermo Fisher Scientific | 15140122 |
| LIVE/DEAD ™ Fixable Near-IR Dead Cell | Thermo Fisher Scientific | L10119 |
| Stain Kit | | |
| Y-27632 (ROCK inhibitor) | Miltenyi Biotec | 130-106-538 |
| Human serum albumin | Albumedix Ltd | 205-005 |
| DPBS without Ca²⁺ and Mg²⁺ | Thermo Fisher Scientific | 14190250 |
| DPBS with Ca²⁺ and Mg²⁺ | Thermo Fisher Scientific | 14040091 |
| StemPro ™ Accutase ™ | Thermo Fisher Scientific | A1110501 |
| Laminin-521 | Biolamina | LN521-05 |
| Poly-L-ornithine | Sigma-Aldrich | P4957 |
| BDNF | R&D Systems | 248-GMP |
| GDNF | R&D Systems | 212-GD |
| Dibutyryl cyclic-AMP (dcAMP) | Sigma-Aldrich | D0627 |
| L-Ascorbic acid | Sigma-Aldrich | A4403 |
| DAPT | Tocris Bioscience | 2634 |
| Transcription Factor Buffer Set | BD Biosciences | 562574 |
| Bovine Serum Albumin | Miltenyi Biotec | 130-091-376 |
| Distilled H₂O | Thermo Fisher Scientific | 10977035 |
| FOXA2-PE antibody (clone: REA506) | Miltenyi Biotec | 130-107-826 |
| SOX2-V450 antibody (clone: O30-678) | BD Biosciences | 561610 |
| Ki67-AF488 antibody (clone: B56) | BD Biosciences | 558616 |
| HuC/HuD aka ELAVL3/ELAVL4 antibody | Invitrogen | A-21271 |
| INA antibody | Atlas Antibodies | HPA008057 |
| SOX2 antibody | R&D Systems | AF2018 |
| FOXA2 antibody | Santa Cruz | Sc-374376 |
| Ki67 antibody | Invitrogen | MA5-14520 |
| TH antibody | Pel-Freez | P40101 |
| Donkey anti-Rabbit IgG AF488 | Invitrogen | A-21206 |
| Donkey anti-Rabbit IgG AF647 | Invitrogen | A-31573 |
| Donkey anti-Mouse IgG AF555 | Invitrogen | A-31570 |

### Example 3: Early In Vitro Administration of MEKi and/or NOTCHi

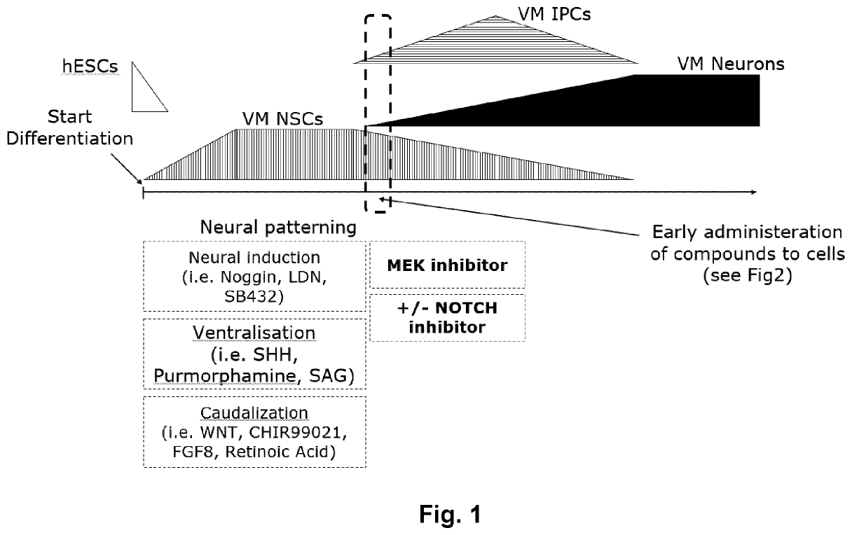

To generate hPSC derived cell cultures or cell products which are enriched in ventral midbrain dopaminergic neurons and depleted in other cell types (i.e., other neurons, glial precursors, glial cells, stromal cells, proliferative cells) novel inhibitors and combinations of inhibitors were administered to ventral midbrain neural cell cultures. Cultures of VM NPCs were exposed to a MEK inhibitor and/or a NOTCH inhibitor immediately after the stages of hPSC neural/ectodermal specification (performed with small molecule SMAD inhibitors, the addition of Noggin), after ventralization (performed with SHH pathway agonists such as SHH, Purmorphamine or SAG) and after caudalization (performed with WNT agonists such as WNT proteins, the small molecule CHIR999021). This exposure immediately after neuroectodermal patterning, ventralization and caudalization is termed “early administration” (FIG. 1). Early administration is when cultures are predominantly comprised of VM NSCs and minimal expression has been observed of VM IPC and/or VM Neuron markers (FIG. 1) corresponding to DIV16 of the protocol in Example 1.

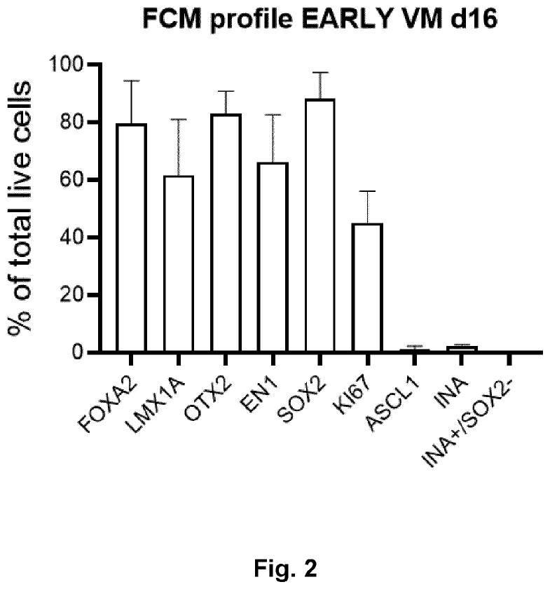

Cells exposed to the early administration of MEK inhibitors and/or NOTCH inhibitors were profiled by flow cytometry (FIG. 2) to assess the protein expression levels of ventral midbrain floor plate regional identity transcription factors (FOXA2, LMX1A, OTX2, EN1) and cell stage markers (KI67 which identifies highly proliferative cells such as NSCs; SOX2 which identifies NSCs; ASCL1 which identifies IPCs; INA which identifies neurons).

TABLE 2
FCM profile VM DIV16

| | FOXA2 | LMX1A | OTX2 | EN1 | SOX2 | Ki67 | ASCL1 | INA | INA+/SOX2− |
|---|---|---|---|---|---|---|---|---|---|
| #1 | 88 | 81.5 | 87.5 | | 95.1 | 40.1 | 2.9 | 3.1 | 0 |
| #2 | 91.3 | 84.0 | 93.6 | 88.2 | 95.6 | 60.3 | 0.7 | 2.6 | 0 |
| #3 | 84.9 | 50.9 | 83.6 | 58.3 | 93.5 | 52.5 | 1.6 | 2.1 | 0 |
| #4 | 80.2 | 46.2 | 76.9 | 68.9 | 83 | 41 | 1.3 | 1.8 | 0 |
| #5 | 53.8 | 46.2 | 74.4 | 50.0 | 75.4 | 32.3 | 0.5 | 1.4 | 0 |
| Mean | 79.7 | 61.8 | 83.2 | 66.3 | 88.5 | 45.2 | 1.4 | 2.2 | 0.000 |
| SEM | 6.71 | 8.63 | 3.49 | 8.243 | 4.00 | 4.95 | 0.42 | 0.30 | 0.000 |

Analyzed cultures were observed to be ventral midbrain in their regional identity, which is shown by their expression of relevant ventral midbrain floor plate markers FOXA2 (>50% of total cells), LMX1A (>45% of total cells) and OTX2 (>70% of total cells) and EN1 (>50% of total cells) (FIG. 2 and Table 2). These cells were still at the NSC stage as shown by their high expression of the NSC marker SOX2 (>70%) and the proliferative marker K167 (>30%). These cultures had begun by the time of exposure to express at low levels markers of ventral midbrain IPCs (ASCL1, 0.48-2.86%) and neurons determined by INA total expression (1.43-3.12%), and not yet express INA+/SOX2− (0%) (FIG. 2 and Table 2).

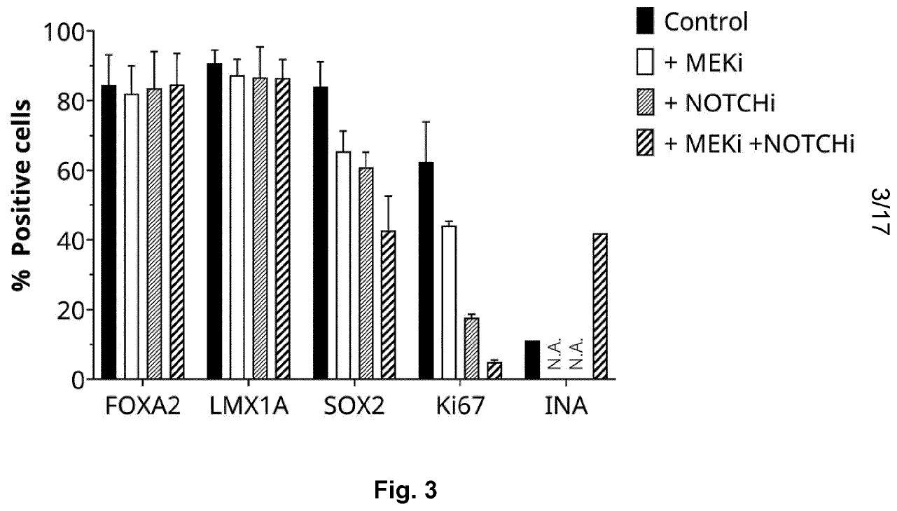

Following 5 days of exposure to MEKi alone, NOTCHi alone or both a MEKi and NOTCHi cultures were re-analyzed using flow cytometry to assess protein expression levels of key genes and compared to untreated controls (FIG. 3 and Table 3).

TABLE 3
FCM results early treatment (DIV16-22)

| | Control | | | | +MEKi | | | |
|---|---|---|---|---|---|---|---|---|
| | #1 | #2 | #3 | #4 | #1 | #2 | #3 | #4 |
| FOXA2 | 82.9 | 73.4 | 88.4 | 93.6 | 81.6 | 74.3 | 90.2 | |
| LMX1A | 91.7 | 85.8 | 95.1 | 90.3 | 88.2 | 82.4 | 91.4 | |
| SOX2 | 79.4 | 78.2 | 84.9 | 93.7 | 60 | 65.1 | 71.5 | |
| Ki67 | 66.4 | 63.5 | 73.3 | 46.1 | 43 | 43.9 | 45.4 | |
| INA | | | | 11.2 | | | | |

| | +NOTCHi | | | | +MEKi + NOTCHi | | | |
|---|---|---|---|---|---|---|---|---|
| | #1 | #2 | #3 | #4 | #1 | #2 | #3 | #4 |
| FOXA2 | 85.3 | 72.4 | 93.2 | | 79.7 | 74.4 | 92.4 | 92 |
| LMX1A | 88.4 | 77.2 | 94.5 | | 85.8 | 80.3 | 93 | 87.2 |
| SOX2 | 59.6 | 57.3 | 65.7 | | 32.4 | 36.4 | 49.1 | 53 |
| Ki67 | 16.6 | 18.6 | 17.7 | | 4.24 | 5.55 | 5.15 | 4.9 |
| INA | | | | | | | | 42 |

All groups were found to maintain high expression (>72.4 of total cells) of the ventral midbrain floor plate lineage markers FOXA2 and LMX1A (FIG. 3, Table 3). Control cells were observed to maintain high expression (78.2-93.7%) of the NSC marker SOX2 and high levels (46.1-73.3%) of the proliferative marker K167 concomitant with low levels of the neuronal marker INA (FIG. 3, black bars). Together this indicates the control cultures were predominantly ventral midbrain floor plate neural stem cells. By contrast, MEKi alone treated cultures (FIG. 3, open bars and Table 3) and NOTCHi alone treated cultures (FIG. 3, narrow striped bars) were both observed to have downregulated expression of the NSC and proliferative markers SOX2 and K167; indicating these cells are differentiating to neurons at a higher rate compared to controls in response to these inhibitors. Most striking observations were made of the combined administration of a MEKi and NOTCHi on cultures (FIG. 3, bold striped bars, and Table 3). Combined administration had decreased to a greater extent the expression of the NSC marker SOX2 (32.4-53%) and dramatically decreased levels of the proliferative marker K167 (4.24-5.55% of all cells), and this was concomitant with a dramatic increase in the expression levels of the neuronal marker INA (42%) (FIG. 3 and Table 3). Together these results indicate that the dual administration of both a MEKi and NOTCHi had a synergizing effect in increasing neuronal and decreasing NSCs and proliferative cell proportions (FIG. 3 and Table 3).

These results support the hypothesis that the early addition of either a MEKi, NOTCHi and more so combined administration of a MEKi and NOTCHi is able to promote the differentiation of ventral midbrain floor plate NSCs into ventral midbrain floor plate neurons without altering the expression of floor plate lineage transcription factors and cell types (FIGS. 1 and 2 and 3, Table 2 and Table 3).

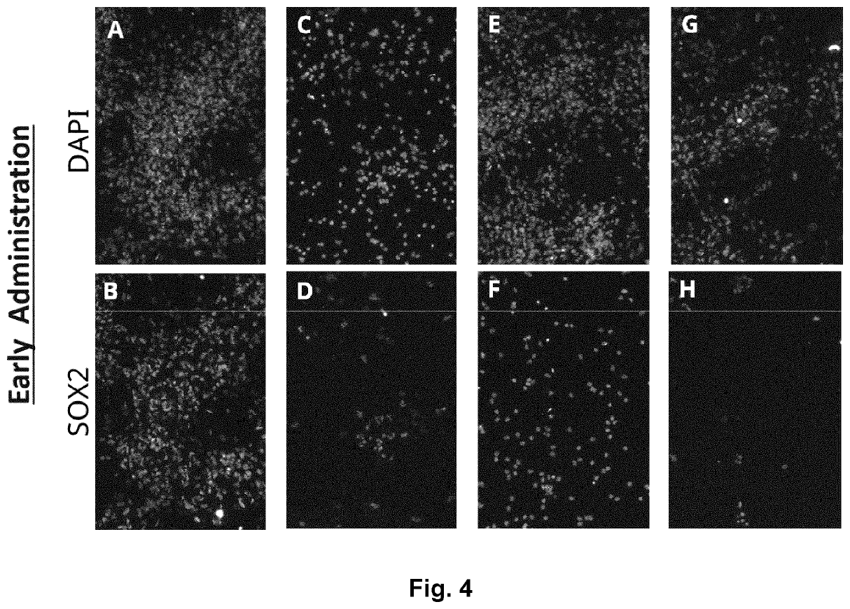

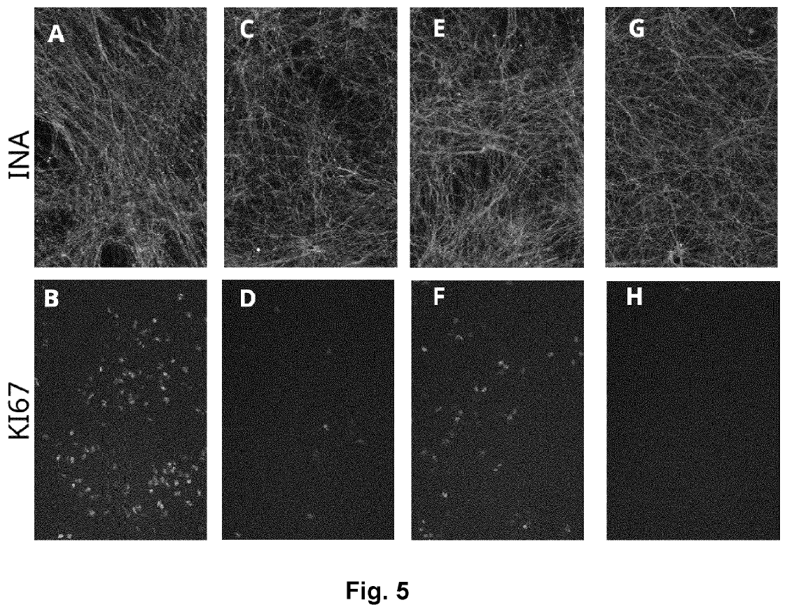

Following compound administration and flow cytometry analysis performed in FIGS. 2 and 3, all groups were differentiated for a further 11 days in vitro and then were fixed in 4% paraformaldehyde and stained with DAPI to identify all cell nuclei and primary antibodies against SOX2 (1:300), Ki-67 (1:250), LMX1A (1:3000), INA (1:200), tyrosine hydroxylase (TH; 1:500) and COL1A1 (1:300) followed by fluorescently labelled secondary antibodies. Images were acquired on an Olympus IX81 microscope using CellSens software. The immunofluorescence (IF) staining confirmed the flow cytometry results of FIG. 3 with extended differentiation showing that treatment with a NOTCHi alone reduced, compared to untreated controls, the amount of NSCs identified by SOX2 (FIG. 4A-D) and proliferative cells identified by KI67 (FIG. 5B,D) while maintaining the amount of neuronal cells identified by INA (FIG. 5A,C).

Also matching earlier time point flow cytometry results, extended differentiation confirmed treatment with a MEKi alone reduced, compared to untreated controls, the amount of NSCs identified by SOX2 (FIG. 4A-B, E-F) and proliferative cells identified by KI67 (FIG. 5B,F) while maintaining the amount of neuronal cells identified by INA (FIG. 5A,E). Further matching earlier time point flow cytometry results, extended differentiation confirmed treatment with a MEKi and NOTCHi reduced, compared to untreated controls, the amount of NSCs identified by SOX2 (FIG. 4A-B, G-H) and proliferative cells identified by KI67 (FIG. 5B,H) while maintaining the amount of neuronal cells identified by INA (FIG. 5A,G).

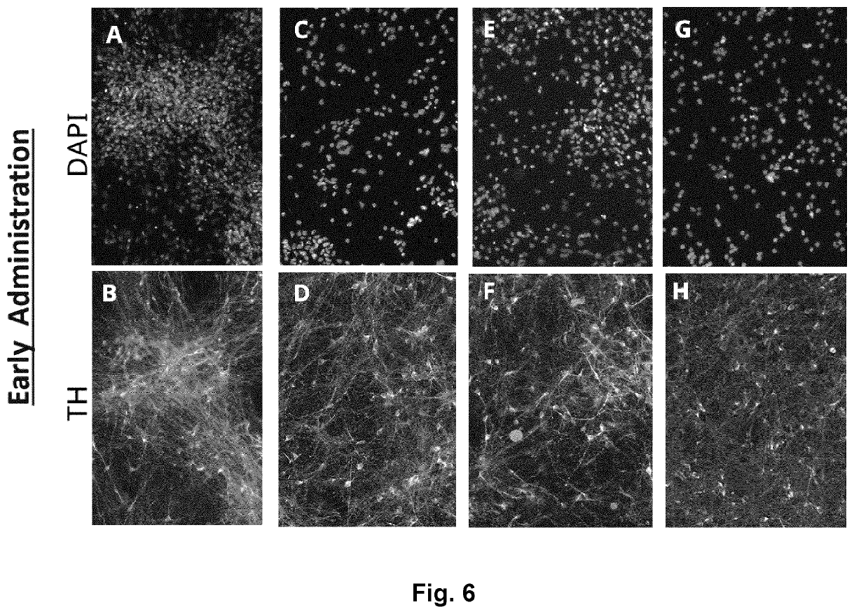

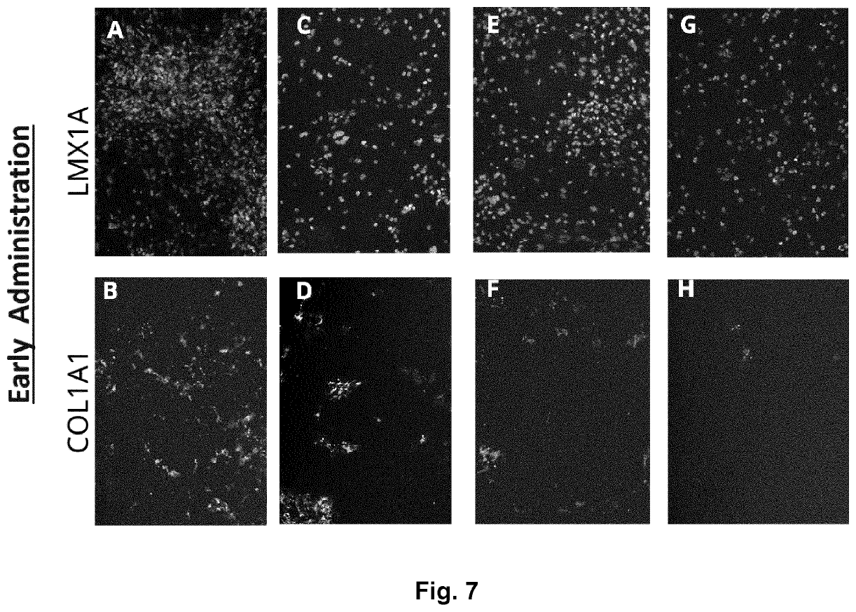

Extended differentiation allowed the analysis of markers of subtype lineages of interest, specifically Tyrosine Hydroxylase (TH) the rate limiting enzyme that identifies dopamine neurons, and COL1A1 a gene expressed by non-neuronal stromal cells. IF staining results of extended differentiations showed that treatment with a NOTCHi alone, treatment with a MEKi alone or dual treatment with both a MEKi and NOTCHi did not prevent the formation of dopamine neurons (FIG. 6B,D,F,H) and comparisons to the number of total cells identified with DAPI shows an increased proportion of TH neurons in inhibitor treated conditions (FIG. 6A-H). IF staining results of extended differentiations also showed that treatment with a NOTCHi alone, treatment with a MEKi alone or dual treatment with both a MEKi and NOTCHi did not prevent the expression of the ventral midbrain lineage marker LMX1A (FIG. 7A,C,E,G). Most remarkably treatment with either a MEKi alone or NOTCHi alone inhibitor dramatically reduced the proportion of stromal COL1A1 cells compared to control untreated cultures (FIG. 7B,D-F) while dual administration of a MEKi and NOTCHi further reduced their proportion (FIG. 7H).

These results support the hypothesis that the early addition of either a MEKi, NOTCH and more so combined administration of a MEKi and NOTCHi is able to promote the differentiation of ventral midbrain floor plate NSCs into ventral midbrain floor plate neurons and specifically dopamine neurons at the expense of non-neuronal cells such as stromal cells without altering the expression of floor plate lineage transcription factors such as LMX1A (FIG. 4-7).

### Example 4: Late In Vitro Administration of MEKi and/or NOTCHi

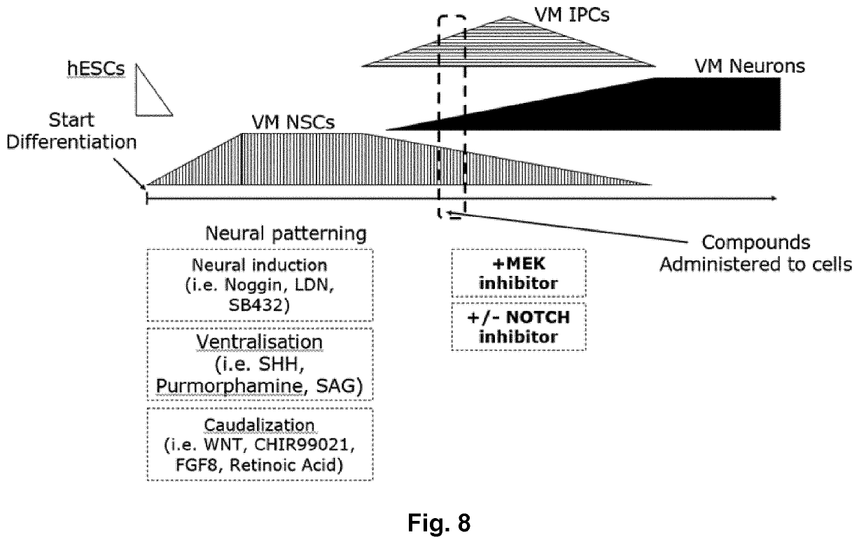

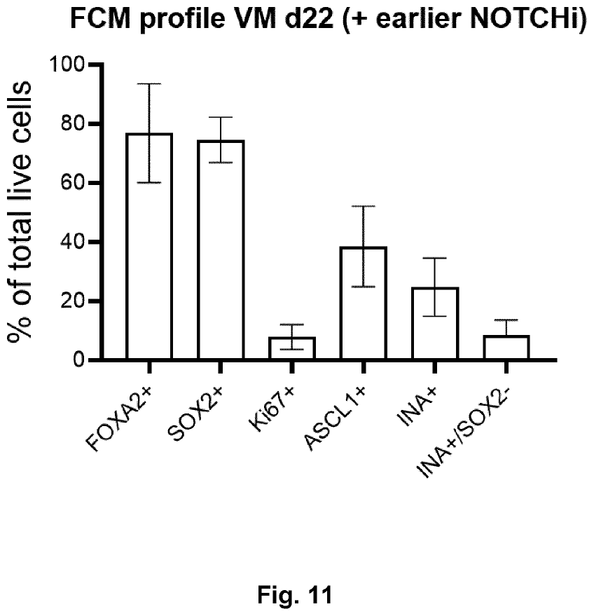

To evaluate the range and capacity of MEKi and/or NOTCHi to promote the differentiation of ventral midbrain floor plate NSCs into ventral midbrain floor plate neurons and specifically dopamine neurons at the expense of non-neuronal cells such as stromal cells, further studies were also performed at later in vitro time points on cultures that had differentiated to a later developmental stage (outlined in FIGS. 8 and 11 and Table 4 and Table 5).

Cultures of VM NPCs that had completed the stages of hPSC neural/ectodermal specification, ventralization and caudalization were left without these patterning factors to further differentiate for a period of time of 6 days in vitro. In this period of time (day in vitro 16-22) and at all points before this (day in vitro 0-22) the cell cultures were not exposed to a MEK inhibitor and/or a NOTCH inhibitor (FIG. 8). In this period of time from day 16-22 cells acquired a greater proportion of cells that were of more mature developmental identities such as IPCs and Neurons, as levels of SOX2 had decreased and increases in the percentage of cells expressing intermediate precursor and neuronal markers ASCL1 and INA and INA+/SOX2− had occurred (FIG. 9, Table 4) compared to the time of early administration in FIGS. 1-2 and Table 2. Cells were then exposed to MEK inhibitors and/or NOTCH inhibitors (FIG. 8) and this was termed “late administration”.

We contemplate this population can be transplanted for the purpose of restoring function in animal models of Parkinson's disease and to human patients for a therapeutic purpose.

TABLE 4
FCM profile VM DIV22 (no earlier NOTCHi)

| | FOXA2 | SOX2 | Ki67 | ASCL1 | INA | INA+/SOX2− |
|---|---|---|---|---|---|---|
| #1 | 83.8 | 90.1 | 52.4 | 42.3 | 13.9 | 2.61 |
| #2 | 90.6 | 91.2 | 60.8 | 61.6 | 13.1 | 3.71 |
| #3 | 89.5 | 92.2 | 56.2 | 58.9 | 12.4 | 3.53 |
| #4 | 63.4 | 79.6 | 50.9 | 44.4 | 13 | 3.3 |
| #5 | 88.4 | 84.9 | 29.1 | 39.1 | 11.8 | |
| Mean | 82.1 | 86.2 | 51.0 | 48.0 | 12.8 | 3.29 |
| SEM | 4.27 | 2.36 | 4.61 | 3.95 | 0.35 | 0.24 |

Previous to late administration, the cells were profiled by flow cytometry (FIG. 9 and Table 4) to assess the protein expression levels of ventral midbrain floor plate regional identity transcription factors (FOXA2) and cell stage markers (NSC and proliferative markers SOX2 and K167; ASCL1 which identifies IPCs; INA which identifies neurons). These analyzed cultures were observed to be ventral midbrain in their regional identity, expressing the ventral midbrain floor plate marker FOXA2 (>60% of all cells) (FIG. 9). These cells were still at the NSC stage as shown by their high expression of the NSC marker SOX2 and K167 (>79% and >50%; FIG. 9, 4). Notably these cultures had matured further than early administration cultures treated in FIG. 1-2, as the levels of ASCL1 have increased dramatically to 48% INA total expression has increased to 12.8% and some cells now are INA+/SOX2− at 3.28%, (FIGS. 9 and 4).

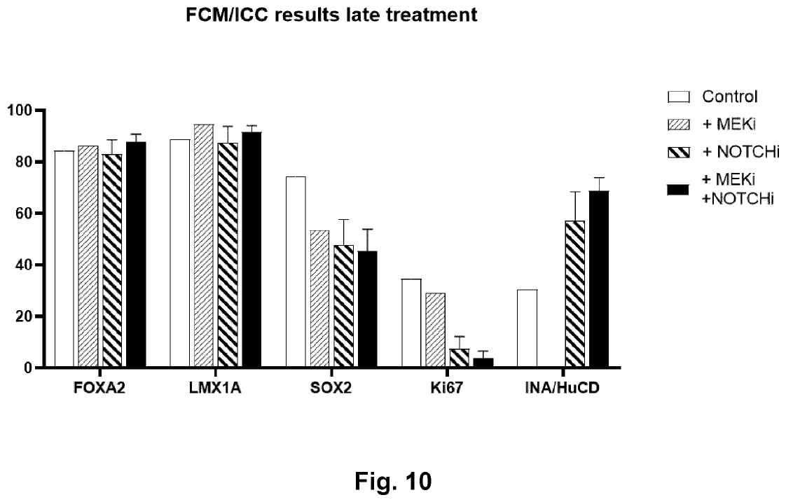

Following 5 days of exposure to MEKi alone, NOTCHi alone or both a MEKi and NOTCHi cultures were re-analyzed using flow cytometry to assess protein expression levels of key genes and compared to untreated controls (FIG. 10, Table 5). Additionally, cultures were differentiated for further 7 days in the absence of any NOTCH inhibitor and ICC was performed with DAPI to identify all cell nuclei and primary antibodies against FOXA2 (1:200), LMX1A (1:1000), SOX2 (1:300), Ki-67 (1:250) and HuC/D (1:100)

TABLE 5
FCM/ICC results late administration (DIV22-29)

| | Control | | | | +MEKi | | | |
|---|---|---|---|---|---|---|---|---|
| | #1 | #2 | #3 | #4 | #1 | #2 | #3 | #4 |
| FOXA2 | 87.4 | 81.9 | 75.2 | 92.6 | | 86.3 | | |
| LMX1A | | 93.6 | 78.9 | 94.2 | | 94.6 | | |
| SOX2 | 80.3 | 57.8 | 81.8 | 77.2 | | 53.4 | | |
| Ki67 | 33.6 | 43.3 | 24.1 | 37.5 | | 29.0 | | |
| INA | 26.4 | | 35.2 | 29.8 | | | | |

| | +NOTCHi | | | | +MEKi + NOTCHi | | | |
|---|---|---|---|---|---|---|---|---|
| | #1 | #2 | #3 | #4 | #1 | #2 | #3 | #4 |
| FOXA2 | 81.8 | 90.2 | 68 | 92.2 | 89.4 | 90.4 | 78.4 | 92.4 |
| LMX1A | | 95.4 | 75.4 | 91.8 | | 95.2 | 86.7 | 92.8 |
| SOX2 | 62.1 | 21.9 | 63.7 | 43.6 | 57.6 | 22.9 | 59.7 | 41.3 |
| Ki67 | 21.3 | 1.65 | 3.87 | 2.98 | 12.3 | 1.37 | 0.80 | 0.36 |
| INA | 35.4 | | 61.5 | 74.1 | 58.7 | | 73.2 | 74.6 |

All groups were found to maintain high expression (of total cells) of the ventral midbrain floor plate lineage markers FOXA2 (>68%) and LMX1A (>80%) (FIG. 10 and Table 5). Control cells were observed to maintain high expression of the NSC marker SOX2 (74% average) and high levels of the proliferative marker KI67 (34.6% average) concomitant with moderate levels of the neuronal marker INA (30.5% average) (FIG. 10, open bars and Table 5). Together this indicates the control cultures were predominantly ventral midbrain floor plate neural stem cells and a subset were neuronal at this later in vitro time point. By contrast, MEKi alone treated cultures (FIG. 10, narrow striped bars and Table 5) and NOTCHi alone treated cultures (FIG. 10, bold striped bars and Table 5) were both observed to have downregulated expression of the NSC and proliferative markers SOX2 and KI67; indicating they cells are differentiating to neurons at a higher rate compared to controls in response to these inhibitors. Combined administration of a MEKi and NOTCHi on cultures (FIG. 10, black/full bars, Table 5) also decreased the expression of the NSC marker SOX2 (45.4% average) and dramatically decreased levels of the proliferative marker KI67 (3.7% of all cells average), and this was concomitant with a dramatic increase in the expression levels of the neuronal marker INA (68.8% average) (FIG. 10, Table 5). Together these results indicate the dual administration of a MEKi, NOTCHi or more so both pathway inhibitors had the effect of increasing neuronal and decreasing NSCs and proliferative cell proportions. Comparisons between the results of treating early versus late VM neural cells with MEKi and NOTCHi shows that late administration resulted in a higher amount of INA neurons (68.8% on average) compared to (42% on average).

These results support the hypothesis that the late addition of a MEKi or NOTCHi or combined administration of a MEKi and NOTCHi is able to promote the differentiation of ventral midbrain floor plate NSCs into ventral midbrain floor plate neurons without altering the expression of floor plate lineage transcription factors. Overall, the data shows that the late administration of the MEKi and NOTCHi is preferable and superior for transitioning VM cells to VM neurons.

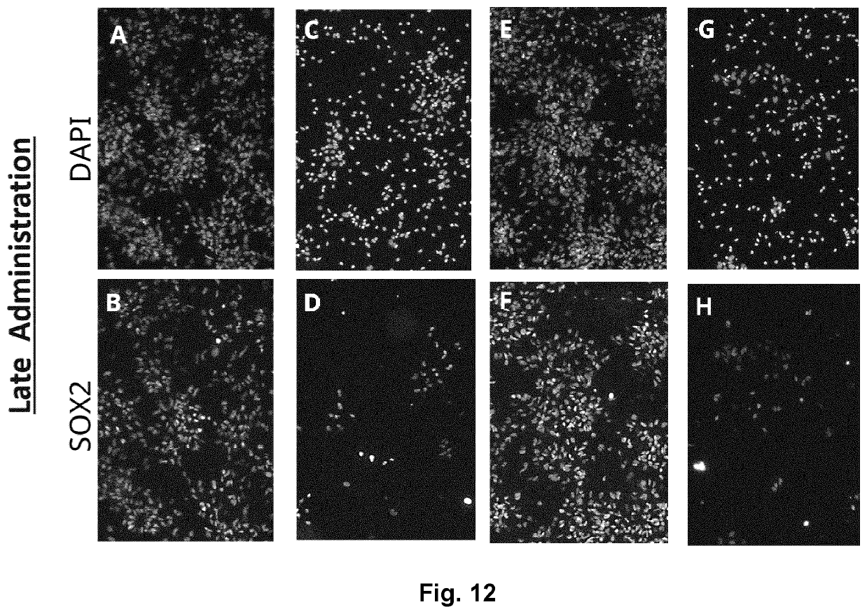

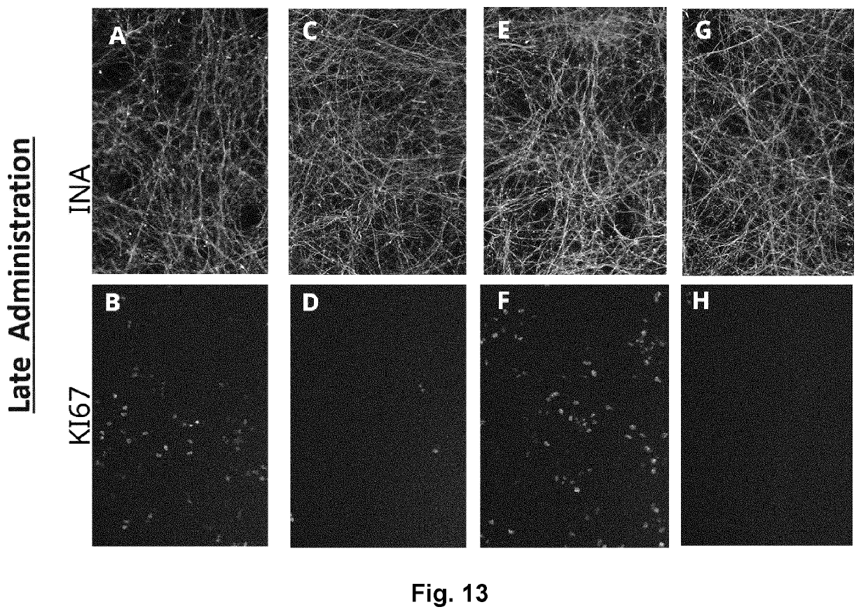

Following compound administration and flow cytometry analysis performed in Figure, all groups were differentiated for a further 11 days in vitro and then were fixed in 4% paraformaldehyde and stained with DAPI to identify all cell nuclei and primary antibodies against SOX2 (1:300), Ki-67 (1:250), LMX1A (1:1000), tyrosine hydroxylase (TH; 1:500) and COL1A1 (1:300) followed by fluorescently labelled secondary antibodies. Images were acquired on an Olympus IX81 microscope using CellSens software. The immunofluorescence (IF) staining confirmed the flow cytometry results with extended differentiation showing that treatment with a NOTCHi alone reduced, compared to untreated controls, the amount of NSCs identified by SOX2 (FIG. 12A-D) and proliferative cells identified by KI67 (FIG. 13B,D) while maintaining the amount of neuronal cells identified by INA (FIG. 13A,C).

Also matching earlier time point flow cytometry results, extended differentiation confirmed treatment with a MEKi alone reduced, compared to untreated controls, the amount of NSCs identified by SOX2 (FIG. 12A-B, E-F) and proliferative cells identified by KI67 (FIG. 13B,F) while maintaining the amount of neuronal cells identified by INA (FIG. 13A,E). Further matching earlier time point flow cytometry results, extended differentiation confirmed treatment with a MEKi and NOTCHi reduced, compared to untreated controls, the amount of NSCs identified by SOX2 (FIG. 12A-B, G-H) and proliferative cells identified by K167 (FIG. 13B, H) while maintaining the amount of neuronal cells identified by INA (FIG. 13A,G).

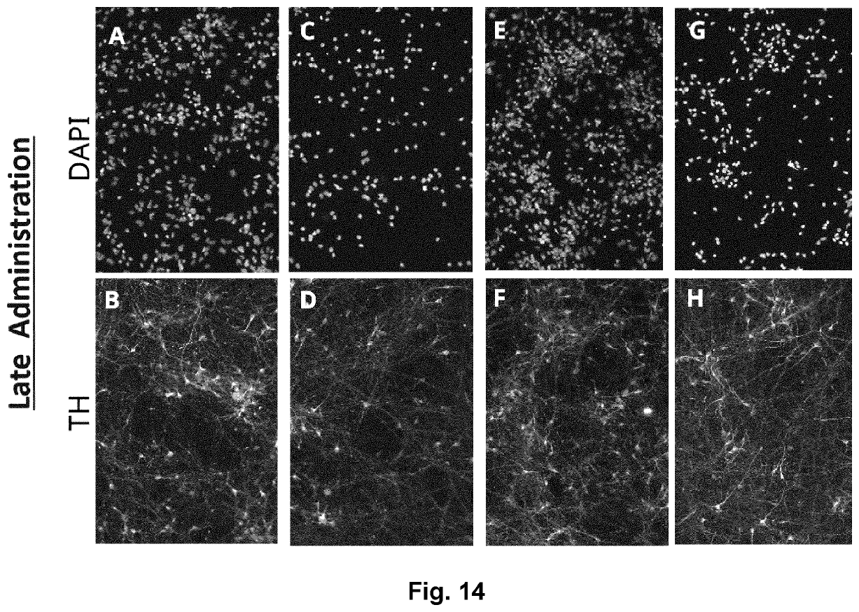

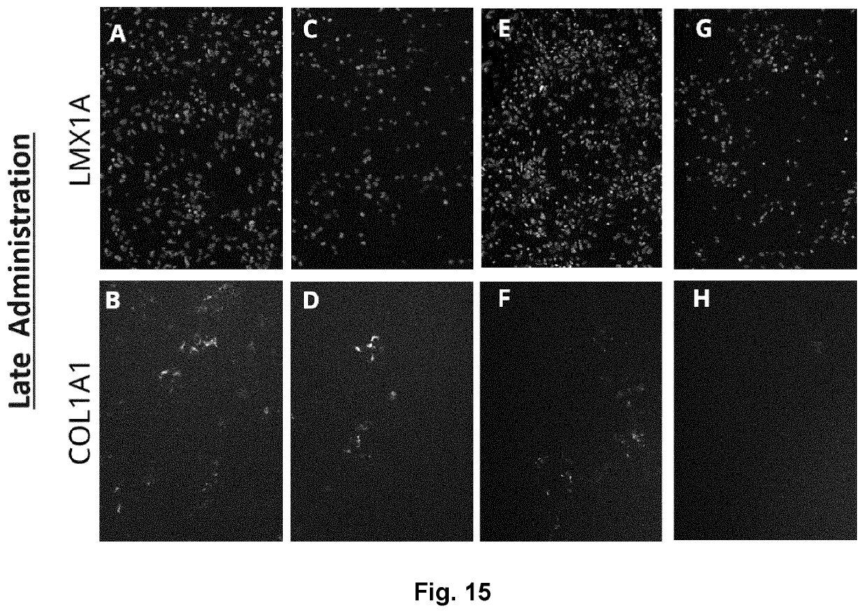

Extended differentiation allowed the analysis of markers of subtype lineages of interest, specifically Tyrosine Hydroxylase (TH) the rate limiting enzyme that identifies dopamine neurons, and COL1A1 a gene expressed by non-neuronal stromal cells. IF staining results of extended differentiations showed that treatment with a NOTCHi alone, treatment with a MEKi alone or dual treatment with both a MEKi and NOTCHi did not prevent the formation of dopamine neurons (FIG. 14B,D,F,H) and comparisons to the number of total cells identified with DAPI shows an increased proportion of TH neurons in inhibitor treated conditions (FIG. 14A-H). IF staining results of extended differentiations also showed that treatment with a NOTCHi alone, treatment with a MEKi alone or dual treatment with both a MEKi and NOTCHi did not prevent the expression of the ventral midbrain lineage marker LMX1A (FIG. 15A,C,E,G). Most remarkably treatment with either a MEKi alone or NOTCHi alone inhibitor dramatically reduced the proportion of stromal COL1A1 cells compared to control untreated cultures (FIG. 15B,D,F) while dual administration of a MEKi and NOTCHi further reduced their proportion (FIG. 15H).

These results support the hypothesis that the late addition of either a MEKi, NOTCHi and more so combined administration of a MEKi and NOTCHi is able to promote the differentiation of ventral midbrain floor plate NSCs into ventral midbrain floor plate neurons and specifically dopamine neurons at the expense of non-neuronal cell such as stromal cells without altering the expression of floor plate lineage transcription factors such as LMX1A (FIG. 12-15).

### Example 5: Profile of Late VM Neural Cells with Earlier Treatment of the NOTCHi from DIV16-22

In this example, cultures of VM NPCs that had completed the stages of hPSC neural/ectodermal specification, ventralization and caudalization were left without these patterning factors to further differentiate from day 16 for a period of time of 6 days in vitro and between DIV16-22 were exposed to a NOTCH inhibitor (DAPT) every 48-72 hrs. Exposing cultures to a NOTCH inhibitor immediately after the cessation of the use of one or more or all patterning factors is typically performed in the field; patterning factors for example are those that are provided to induce neuroectoderm of the embryo (i.e. NOGGIN, SMAD inhibitors) and/or dorso-ventral patterning factors (i.e. BMPs, SHH, SAG, Purmorphamine) and/or rostro-caudal patterning factors (i.e. WNT proteins, CHIR, FGF8), as described in publications Nolbrant et al., 2017, Kriks et al. 2011, Nishimura et al., 2023. In this period of time (DIV16-22) and at all points before this (day in vitro 0-16) the cell cultures were not exposed to a MEK inhibitor.

This addition of NOTCH inhibition immediately after patterning factors are removed at DIV16 when cultures are predominantly NPCs and do not comprise or comprise <2% INA+/SOX2− cells (FIG. 2, Table 1) and until DIV22 leads to a profound impact on the cell stage/maturity meaning the proportion of IPCs and neurons at DIV22. This is observable at the protein level via flow cytometry profile, see FIG. 9 and Table 4 showing the profile at DIV22 without any earlier NOTCH inhibition and compared to FIG. 11 and Table 6 with earlier NOTCHi. Notably, earlier (DIV16-22) treatment with NOTCH inhibition leads to a higher proportion of neurons as INA+ cells (24.7% with NOTCHi vs 12.8% without) or INA+/SOX2-cells (8.4% with NOTCHi vs 3.3% without). As such we contemplate this early treatment with a NOTCHi immediately after withdrawal of patterning factors leads to a less optimal culture for exposure to dual MEKi/NOTCHi treatment and conversion to neurons as there are more cells already at the neuronal stage. Further we anticipate this population with more neurons is less optimal for transplantation (as neurons are fragile and sensitive to transplantation stress) and we anticipate that the population of FIG. 9 and Table 4 that comprises many ASCL1+IPCs (51.8%) with minimal neuronal (INA+ or INA+/SOX2−) cells is more optimal for transplantation and/or addition of dual MEK and NOTCH inhibition.

### REFERENCES

Kaneyasu Nishimura, Shanzheng Yang, Ka Wai Lee, Emilia Sif Asgrimsdottir, Kasra Nikouei, Wojciech Paslawski, Sabine Gnodde, Guochang Lyu, Lijuan Hu, Carmen Salto, Per Svenningsson, Jens Hjerling-Leffler, Sten Linnarsson, and Ernest Arenas. Single-cell transcriptomics reveals correct developmental dynamics and high quality midbrain cell types by improved hESC differentiation. 2023. Stem Cell Reports. https://doi.org/10.1016/i.stemcr.2022.10.016
Sonja Kriks, Jae-Won Shim, Jinghua Piao, Yosif M Ganat, Dustin R Wakeman, Zhong Xie, Luis Carrillo-Reid, Gordon Auyeung, Chris Antonacci, Amanda Buch, Lichuan Yang, M Flint Beal, D James Surmeier, Jeffrey H Kordower, Viviane Tabar, Lorenz Studer. Dopamine neurons derived from human ES cells efficiently engraft in animal models of Parkinson's disease. 2011. Nature. 10.1038/nature10648

TABLE 6
FCM profile VM DIV22 (+earlier NOTCHi)

| | FOXA2 | SOX2 | Ki67 | ASCL1 | INA | INA+/SOX2− |
|---|---|---|---|---|---|---|
| #1 | 85.4 | 72 | 5.9 | 50.4 | 23.6 | 7.3 |
| #2 | 98.2 | 70.5 | 4.8 | 57.1 | 34.2 | 13.6 |
| #3 | 82.1 | 64.7 | 6.0 | 40.5 | 19 | 5.7 |
| #4 | 94.9 | 65.7 | 6.6 | 51.8 | 45.6 | 20.1 |
| #5 | 67.6 | 69.5 | 6.1 | 36.0 | 18.3 | 7.0 |
| #6 | 69.7 | 70.9 | 6.4 | 41.1 | 30.5 | 13.8 |
| #7 | 76.2 | 86.3 | 8.1 | 41.7 | 19.2 | 4.7 |
| #8 | 82.7 | 88.3 | 9.7 | 45.8 | 22.4 | 6.2 |
| #9 | 78.0 | 77.2 | 7.8 | 16.2 | 13.8 | 2.6 |
| #10 | 75.5 | 78.5 | 5.2 | 17.3 | 13.3 | 5.0 |
| #11 | 35.2 | 76.4 | 19.8 | 25.6 | 32.0 | 6.0 |
| Mean | 76.9 | 74.5 | 7.9 | 38.5 | 24.7 | 8.4 |
| SEM | 5.04 | 2.32 | 1.27 | 4.11 | 2.97 | 1.58 |

### Example 6: Further Culturing the Cell Population after MEK/NOTCH Inhibition to Assess Expression Profile

After administration with MEK and NOTCH inhibitors, cells are cultured for a further 5 days in a 2D culture in wells coated with poly-L-ornithine (0.002%) and Laminin-521 (1.5 μg/cm2) in neural supportive media supplemented with BDNF (20 ng/mL), GDNF (20 ng/mL), L-ascorbic acid (200 μM), dcAMP (500 μM), or other such supportive media and this time allows time for the transition of neural precursors and intermediates and to terminally differentiate into ventral midbrain neurons. After 5 days of culture the expression profile of the cell population can be assessed according to any one of the methods described in Example 7, Example 8, and Example 9.

### Example 7: RNA Sequencing Methodology

To perform single cell RNA sequencing (scRNA-seq) or single nuclei RNA sequencing, undifferentiated PSCs as well as those of differentiated cells were dissociated into single cell suspensions with accutase, tryple select or other such reagents and 3000-10000 cells were processed using the 10× Genomics Chromium Platform and sequenced on a NextSeq550. Data was processed using 10× cellranger and the Seurat analysis package in R programming language. Samples were analyzed, filtered for low quality or multiplet cells and analyzed separately for each individual experiment before combining the cells of the selected differentiated cell lineages of choice as well as the hPSCs into one dataset that were then analyzed using the standard Seurat workflow as outlined for Seurat version 3, i.e., normalizing using SCTransform and finally using the first 29 principal components for the unified tSNE plots.

### Example 8: Immunocytochemistry (ICC)

Cells were fixed in 4% paraformaldehyde (Alfa Aesar) for 10 minutes at room temperature. Unspecific antibody binding was blocked by incubating cells with PADT buffer; phosphate-buffered saline (PBS) without Ca2+ and Mg2+ (Gibco) with 0.02% sodium azide solution (Ampliqon), 0.5% Triton X-100 (Sigma), and 5% Donkey serum (Jackson Labs) for 30 minutes, followed by overnight incubation with primary antibodies (see Table X) at 4° C. The cells were washed 3 times with PBS without Ca2+ and Mg2+, blocked with PADT buffer for 15 minutes, and incubated with fluorophore-conjugated secondary antibodies (see Table X) for 2 hours at room temperature, protected from light. The cells were then counterstained with DAPI (10 μg/mL) for 5 minutes at room temperature, washed 3 times with PBS without Ca2+ and Mg2+, and stored at 4° C. in PBS without Ca2+ and Mg2+ supplemented with 0.02% sodium azide. Images were captured with a Zeiss Axio Observer microscope equipped with an Axiocam 512 camera and ZEN 3.2 (Pro) software (Zeiss).

### Example 9: Quantitative Real Time PCR (qPCR, or qRT-PCR)

To perform qPCR RNA is extracted from cells with Trizol and converted to cDNA and subsequently analyzed using quantitative real-time polymerase chain reaction (qPCR) for genes of interest such as SOX2, KI67, HuCD, NeuN, INA, ASCL1, FOXA2, TH, LMX1A, EN1 or other relevant markers for ventral midbrain neural cells. qPCR is typically performed across triplicate technical replicates for each of 3 or more independent biological replicates and normalized against housekeeping genes such as GAPDH or HPRT1.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An in vitro method for directing differentiation of a cell population of PSCs into ventral midbrain neurons, comprising culturing the cell population of PSCs, contacting the cell population of PSCs with an inhibitor of SMAD protein signaling, an inhibitor of Wnt signaling, an activator of SHH signaling, an activator of FGF signaling, optionally ascorbic acid, and, optionally BDNF, to obtain a cell population comprising ventral midbrain NSCs, wherein the cell population comprising ventral midbrain NSCs is further contacted with an inhibitor of MEK signaling and an inhibitor of NOTCH signaling to direct differentiation into ventral midbrain neurons, and wherein the cell population is allowed to differentiate into ventral midbrain NSCs for 14 to 24 days prior to contacting the cell population with the inhibitor of MEK signaling and the inhibitor of NOTCH signaling.

2. An in vitro method for directing differentiation of ventral midbrain NSCs into neurons comprising contacting a cell population comprising ventral midbrain NSCs with an inhibitor of MEK signaling and an inhibitor of NOTCH signaling, wherein at least 5% of the cell population comprising ventral midbrain NSCs co-express the markers FOXA2, LMX1A, EN1, OTX2, and SOX2, and wherein the cell population is allowed to differentiate into ventral midbrain NSCs for 14 to 24 days prior to contacting the cell population with the inhibitor of MEK signaling and the inhibitor of NOTCH signaling.

3. An in vitro method for directing differentiation of ventral midbrain NSCs into neurons comprising contacting a cell population comprising ventral midbrain NSCs with an inhibitor of MEK signaling and an inhibitor of NOTCH signaling, wherein 40-60% of the cell population comprising ventral midbrain NSCs express ASCL1, 45-65% of the cell population comprising ventral midbrain NSCs express KI67, 10-15% of the cell population comprising ventral midbrain NSCs express INA, 2-5% of the cell population comprising ventral midbrain NSCs are INA+/SOX2−, and 80-95% of the cell population comprising ventral midbrain NSCs express SOX2.

4. The method according to claim 1, comprising initially allowing differentiation of the cell population into ventral midbrain NSCs, wherein the cell population is harvested at day 28, day 27, day 26, or day 25 from initially allowing differentiation of the cell population into ventral midbrain NSCs, preferably at day 25.

5. The method according to claim 1, wherein the inhibitor of MEK signaling is selected from PD0325901, trametinib (GSK1120212), selumetinib (AZD6244), pimasertib (AS703026), MEK162, cobimetinib, PD184352, PD173074, BIX02189, AZD8330, PD318088, Refametinib, and PD98059, preferably PD0325901.

6. The method according to claim 1, wherein the inhibitor of NOTCH signaling is selected from DAPT, Avagacestat, PF-03084014, and LY450139.

7. The method according to claim 1, wherein the cell population is contacted with the inhibitor of MEK signaling and the inhibitor of NOTCH signaling for at least at least ½ hour, 1 hour, 2, 3, 4, 5, 6, 7, 8, 9, 10, 11, or 12 hours, or 18 hours, or 24 hours, or for at least 2 days, 3 days, 4 days, 5 days, or 6 days.

8. The method according to claim 1, wherein the cell population is contacted with the inhibitor of MEK signaling and the inhibitor of NOTCH signaling at least partly at the same time.

9. The method according to claim 1, wherein the cell population is contacted with the inhibitor of MEK signaling and the inhibitor of NOTCH signaling simultaneously.

10. The method according to claim 9, wherein the cell population is cryopreserved within 4 days following end of inhibition of MEK signaling and inhibition of NOTCH signaling.

11. An in vitro cell population comprising ventral midbrain NSCs, wherein the cell population when cultured in vitro for 5 days in a culture medium suitable for maintaining neural cells results in a cell population wherein at least 50% are INA+ or HuCD+, and less than 15% are K167+.

12. The cell population according to claim 11, wherein the cell population is cultured according to Example 6.

13. The cell population according to claim 11, wherein the expression of markers by the cells is measured according to the method described in Example 8.

14. A composition comprising a cell population comprising ventral midbrain NSCs, an inhibitor of MEK signaling, and an inhibitor of NOTCH signaling.

15. The composition according to claim 14 for the treatment of Parkinson's disease.
